(19) United States
(12) Patent Application Publication
(10) Pub. No.: US 2006/0142188 A1
(43) Pub. Date: Jun. 29, 2006

(54) Monkey alpha-7 nicotinic acetylcholine receptor and methods of use thereof

(75) Inventors: Shaojie Wang, Paramus, NJ (US); Daguang Wang, New York, WY (US)

Correspondence Address: DARBY & DARBY P.C., P. O. BOX 5257, NEW YORK, NY, 10150-5257, US

(73) Assignee: Memory Pharmaceuticals Corporation, Montvale, NJ (US)

(21) Appl. No.: 10/545,334

(22) PCT Filed: Feb. 13, 2004
(86) PCT No.: PCT/US04/04364

§ 371 Date: Aug. 10, 2005

Related U.S. Application Data
(60) Provisional application No. 60/447,288, filed on Feb. 14, 2003.
(60) Provisional application No. 60/453,204, filed on Mar. 11, 2003.

(51) Int. Cl. A61K 38/17; C07K 14/705; C07H 21/04; C12P 21/06; C12N 5/06

(52) U.S. Cl. 514/12; 530/350; 435/69.1; 435/320.1; 435/362; 536/23.5

(57) ABSTRACT

Monkey alpha-7 neuronal nicotinic acetylcholine receptor polypeptides, as well as the DNA (RNA) encoding such polypeptides, are disclosed. Also disclosed are methods for utilizing such polypeptides in diagnostic assays for identifying mutations in nucleic acid sequences encoding the polypeptides of the present invention, for detecting altered levels of the polypeptide of the present invention as a means of detecting diseases and methods of screening potential modulators of the novel alpha-7 receptor disclosed herein. Transgenic animals expressing polypeptides disclosed herein are also described.

The present application claims the benefit of U.S. Provisional Application Ser. Nos. 60/447,288, filed Feb. 14, 2003, and 60/453,204, filed Mar. 11, 2003, which are hereby incorporated by reference in their entirety.

### BACKGROUND OF THE INVENTION

There are two types of receptors for the neurotransmitter, acetylcholine: muscarinic receptors and nicotinic receptors, based on the selectivity of action of muscarine and nicotine, respectively. Muscarinic receptors are G-protein coupled receptors. Nicotinic receptors are members of the ligand-gated ion channel family. When activated, the conductance of ions across the nicotinic ion channels increases.

Nicotinic alpha-7 receptor protein forms a homo-pentameric channel in vitro that is highly permeable to a variety of cations (e.g., Ca⁺⁺). Each nicotinic alpha-7 receptor has four transmembrane domains, named M1, M2, M3, and M4. The M2 domain has been suggested to form the wall lining the channel. Sequence alignment shows that nicotinic alpha-7 is highly conserved during evolution. The M2 domain that lines the channel is identical in protein sequence from chicken to human. For discussions of the alpha-7 receptor, see, e.g., Revah et al. (1991), Nature, 353, 846-849; Galzi et al. (1992), Nature 359, 500-505; Fucile et al. (2000), PNAS 97(7), 3643-3648; Briggs et al. (1999), Eur. J. Pharmacol. 366 (2-3), 301-308; and Gopalakrishnan et al. (1995), Eur. J. Pharmacol. 290(3), 237-246.

The nicotinic alpha-7 receptor channel is expressed in various brain regions and is believed to be involved in many important biological processes in the central nervous system (CNS), including learning and memory. Nicotinic alpha-7 receptors are localized on both presynaptic and postsynaptic terminals and have been suggested to be involved in modulating synaptic transmission. Nicotinic alpha-7 receptors are therefore of interest in drug development of compounds that modulate the activity of neuronal nicotinic alpha-7 receptors.

### BRIEF DESCRIPTION OF THE DRAWINGS

FIG. 1: Nucleotide sequence for a cDNA encoding for rhesus monkey alpha-7 nACh receptor subunit. (SEQ ID NO: 1).The start codon ATG and stop codon TAA are indicated in bold.

FIG. 2: Amino acid sequence for rhesus monkey alpha-7 nACh receptor subunit. (SEQ ID NO: 2)

FIG. 3: Nucleotide sequence for a cDNA encoding for mutant (L270T) rhesus monkey alpha-7 nACh receptor subunit. (SEQ ID NO: 3) The start codon ATG and stop codon TAA are indicated in bold.

FIG. 4: Amino acid sequence for mutant (L270T) rhesus monkey alpha-7 nACh receptor subunit. (SEQ ID NO: 4)

FIG. 5: Nucleotide sequence for a cDNA encoding for double-mutant (L270T/S193N) for rhesus monkey alpha-7 nACh receptor subunit. The start codon ATG and stop codon TAA are indicated in bold. (SEQ ID NO: 5)

FIG. 6: Amino acid sequence for double-mutant (L270T/S193N) for rhesus monkey alpha-7 nACh receptor subunit. (SEQ ID NO: 6)

FIG. 7: Functional dose-dependent response of nicotine and GTS-21 to mutant (L270T) alpha7 nAchR stably expressed in QM7 cells. The experiment was measured with FLIPR. The EC50 of GTS-21, a specific alpha7 nAchR agonist, is better or comparable to that of nicotine.

FIG. 8: Dose response curve of rhesus monkey wild-type alpha-7 clone #1 to acetylcholine (Ach). The EC₅₀ of this receptor is 38 μM, which is comparable to EC₅₀s for human and rat wild type alpha-7 receptors (21 μM and 28 μM respectively; Papke, R. L. and Porter, J. K. (2002) Comparative pharmacology of rat and human alpha7 nAChR conducted with net charge analysis. Br. J. Pharmacol. 137(1), 49-61.)

FIG. 9: Dose-response curve for double-mutant (L270T/S193N) for rhesus monkey alpha-7 nACh receptor subunit using GTS-21.

### DESCRIPTION OF THE INVENTION

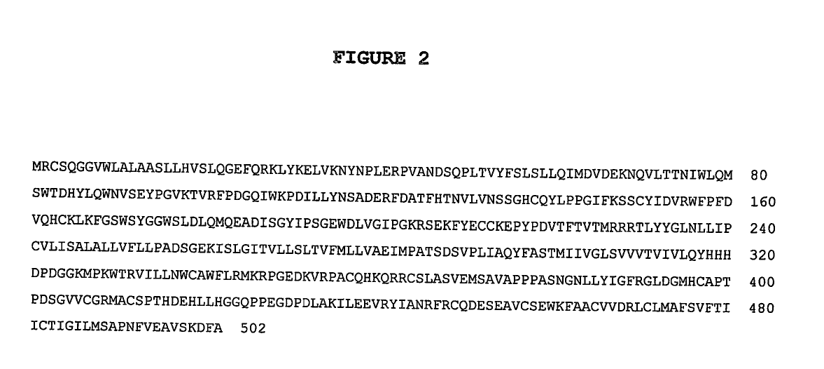

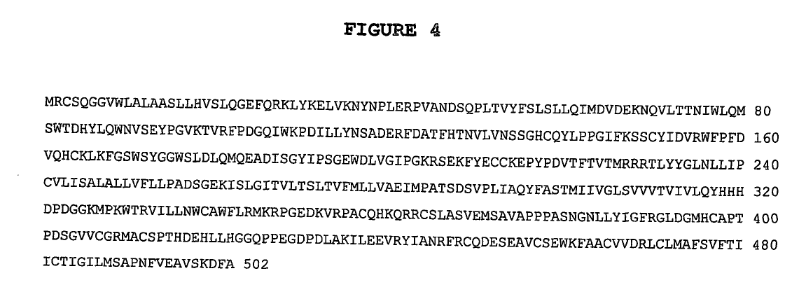

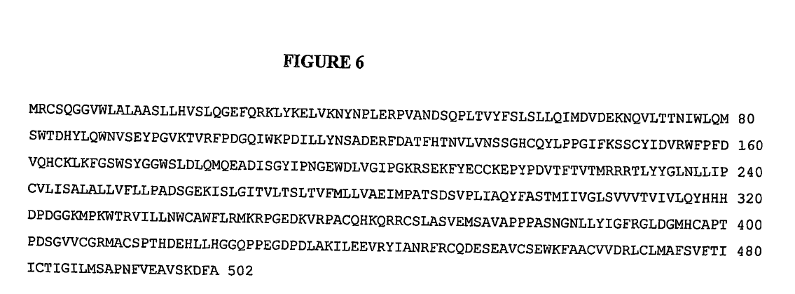

The present invention relates to monkey alpha-7 neuronal nicotinic acetylcholine receptor (“alpha-7 receptor”), variants, fragments thereof, antibodies thereto, their uses, etc. One aspect of the invention is an isolated full-length rhesus monkey alpha-7 receptor protein, as represented by FIG. 2 (SEQ ID NO: 2) (wild-type), and mutations to it, such as amino acid substitutions in the M2 domain. Examples of variants, include, e.g., the polypeptide sequences shown in FIGS. 4 and 6 (SEQ ID NOS:4 and 6). The polypeptides represented by these sequences each have 502 amino acids.

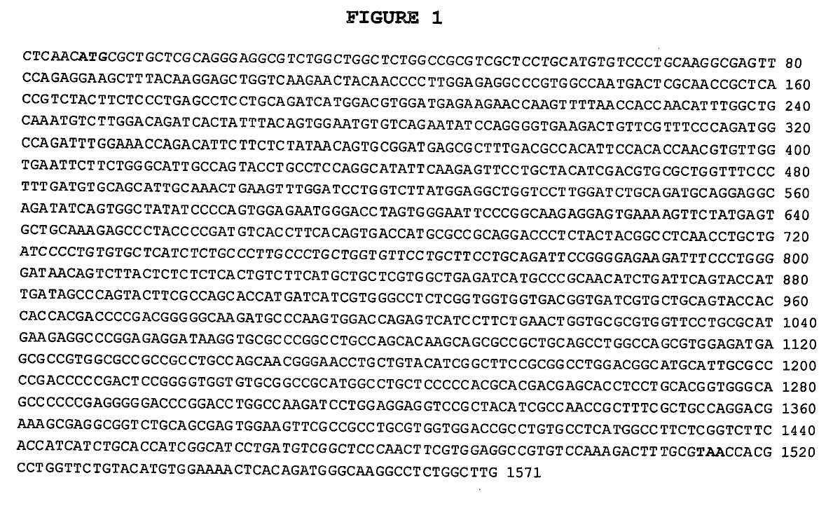

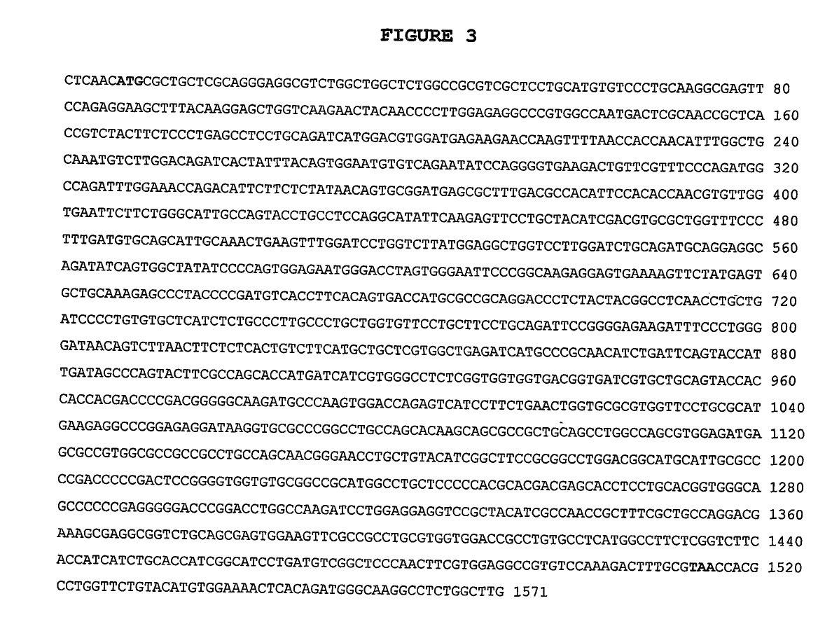

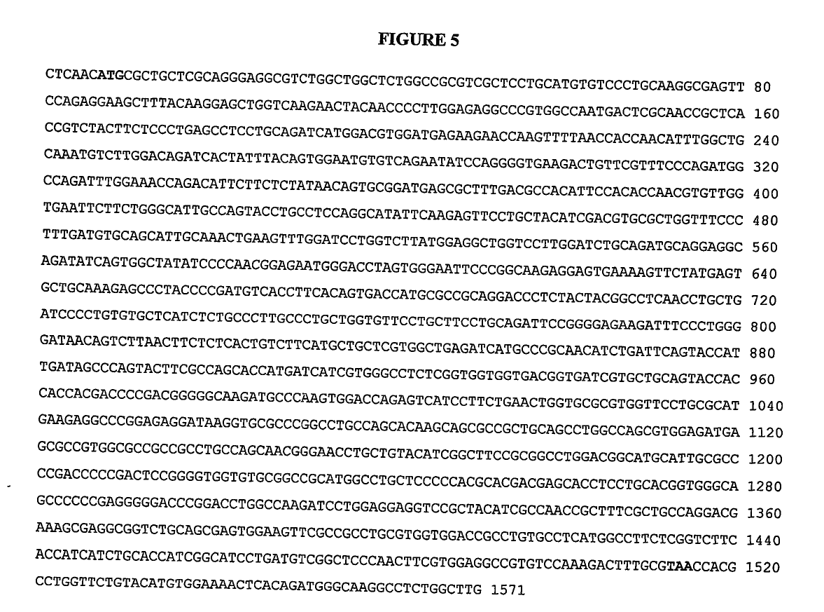

Another aspect of the invention is an isolated cDNA, which encodes a full-length rhesus monkey alpha-7 receptor protein. Typical cDNAs are represented by SEQ ID NO: 1 (FIG. 1; wild-type) and SEQ ID NOS: 3 and 5 (FIGS. 3 and 5; mutant). The plasmid MKALPHA7 containing the cDNA of SEQ ID NO:1 was deposited on Feb. 13, 2003, with the American Type Culture Collection (ATCC), 10801 University Blvd., Manassas, Va. 20110-2209, U.S.A. under the provisions of the Budapest Treaty for the International Recognition of the Deposit of Microorganisms for the Purposes of Patent Procedure and was accorded ATCC Accession No. PTA-5004.

Thus, the invention relates, e.g., to an isolated polynucleotide comprising the cDNA sequence of FIG. 1, 3, or 5 (SEQ ID NOS 1,3, or 5). The invention also relates to an isolated polynucleotide comprising a fragment or variant of these sequences, or complements thereto.

Another aspect of the invention is an isolated polynucleotide which comprises a nucleotide sequence that codes without interruption for the polypeptide of FIG. 2, 4, or 6 (SEQ ID NO: 2, 4, or 6) or a fragment, variant, or complement thereof. A polynucleotide which “codes without interruption” refers to a polynucleotide having a continuous open reading frame (“ORF”) as compared to an ORF which is interrupted by introns or other noncoding sequences.

The invention also relates to methods of making the above-described polypeptides and fragments thereof, or polynucleotides. The methods include,e.g., methods of making constructs which comprise and/or express the polynucleotide sequences; and methods of transforming cells with constructs capable of expressing the polypeptides, culturing the transformed cells under conditions effective to express the polypeptides, and harvesting (recovering) the polypeptides; to antibodies, antigen-specific fragments, or other specific binding partners which are specific (selective) for the polypeptides; to methods of detecting a disease condition or a susceptibility to a disease condition that is associated with aberrant expression (e.g., under- or over-expression) of the polypeptides or polynucleotides, or with variant forms (e.g., mutants, polymorphisms, SNPs, etc.) of the polypeptides or polynucleotides; to methods of treating such disease conditions (e.g., any of a variety of memory dysfunctions) or of stimulating memory formation; to methods of using polypeptides, polynucleotides or antibodies of the invention to detect the presence or absence, and/or to quantitate the amounts, of the polypeptides and polynucleotides of the invention in a sample; to methods of detecting mutations in the polypeptide or polynucleotide sequences which are associated with a disease condition; to methods of using the polypeptides or polynucleotides, or cells transformed with the polynucleotides, to screen for potential therapeutic agents, e.g., agents which modulate the activity or amounts of the polynucleotides or polypeptides; to transgenic animals which express the polypeptides or knockout animals which do not express the polypeptides; or for other potential uses.

For example, the invention relates to an isolated polypeptide, comprising the amino acid sequence of FIG. 2, 4, or 6 (SEQ ID NOS: 2, 4, or 6), or a fragment or variant thereof. The polypeptide may comprise, e.g., at least about 10, 12, 14, 15, 20, 25, 50, 100, 200, etc., contiguous amino acids of FIG. 2, 4, or 6 (SEQ ID NOS: 2, 4, or 6) and/or may have a sequence identity of, e.g., at least about 65%, 70-75%, 80-85%, 90-95% or 97-99% to FIG. 2, 4, or 6 (SEQ ID NOS: 2, 4, or 6) or a fragment thereof; and/or may comprise a sequence that is substantially homologous to FIG. 2, 4, or 6 (SEQ ID NOS: 2, 4, or 6) or a fragment thereof; and/or may be encoded by cDNA contained in ATCC Deposit No PTA-5004, or a fragment thereof. The polypeptide may further comprise a heterologous sequence; may exhibit alpha-7 receptor activity; may be from a mammal, and/or may be substantially purified. The polypeptide may have the amino acid sequence of FIG. 2, 4, or 6 (SEQ ID NOS: 2, 4, or 6).

In another aspect, the invention relates to an isolated polynucleotide which comprises the nucleotide sequence of FIG. 1, 3, or 5 (SEQ ID NOS: 1, 3, or 5) or a fragment or variant of FIG. 1, 3, or 5 (SEQ ID NOS: 1, 3, or 5) or a complement thereof The polynucleotide many comprise; e.g., at least about 8, 10, 12, 14, 15, 20, 25, 30, 50, etc., contiguous nucleotides of FIG. 1, 3, or 5 (SEQ ID NOS: 1, 3, or 5), e.g., about 15 continuous nucleotides. The polynucleotide may further comprise a heterologous sequence; and/or may be from a mammal, and/or may be DNA, cDNA, RNA, PNA or combinations thereof. The polynucleotide may have a nucleotide sequence of the cDNA contained in ATCC Deposit No. PTA-5004 or of a fragment thereof; and/or may comprise a sequence that hybridizes to FIG. 1, 3, or 5 (SEQ ID NOS: 1, 3, or 5) or a fragment thereof under conditions of high stringency; and/or may comprise a sequence that is substantially homologous to FIG. 1, 3, or 5 (SEQ ID NOS: 1, 3, or 5) or a fragment thereof; and/or may have a sequence identity of, e.g., at least about 65%, 70-75%, 80-85%, 90-95% or 97-99% to FIG. 1, 3, or 5 (SEQ ID NOS: 1, 3, or 5) or a fragment thereof; and/or may have the nucleotide sequence of FIG. 1, 3, or 5 (SEQ ID NOS: 1, 3, or 5).

In another aspect, the invention relates to a recombinant construct comprising a polynucleotide as above, which may be operatively linked to a regulatory sequence, e.g., wherein said construct comprises a baculovirus expression vector. The invention also relates to a cell comprising such a construct, e.g., a mammalian, human, yeast, QM7, QT6, or insect cell, preferably an SF9 cell. The invention also relates to a method of making such a cell, comprising introducing a construct or polynucleotide as above into a cell. The invention also relates to a method to make a polypeptide of the invention, comprising incubating a cell as above under conditions in which the polypeptide is expressed, and harvesting the polypeptide.

In another aspect, the invention relates to an antibody, antigen-specific antibody fragment, or other specific binding partner, which is specific for a polypeptide of the invention, e.g., wherein said antibody, antigen-specific antibody fragment, or specific binding partner is specific for the polypeptide of SEQ ID NO: 2 or 4 or a fragment or variant thereof.

In another aspect, the invention relates to methods of diagnosis, e.g., a method to determine the presence of a disease condition or a susceptibility to a disease condition in a patient in need thereof, where said condition is associated with an over- or underexpression of a polynucleotide (e.g., mRNA) of the invention, comprising contacting a cell, tissue, cell extract, or nucleic acid of said patient with a polynucleotide as above, and/or determining the amount or level of said nucleic acid. The cell or nucleic acid may be from the brain of said patient, e.g., from the hippocampus, and may be from a neuron.

The invention also relates to a method of diagnosis, comprising determining a mutation or polymorphism or SNP in the genome of a cell, wherein said mutation occurs in the nucleotide sequence of FIG. 1, 3, or 5 (SEQ ID NOS: 1, 3, or 5), or in the sequence of a polynucleotide which encodes a polypeptide of FIG. 2, 4, or 6 (SEQ ID NOS: 2, 4, or 6).

The invention also relates to a method to determine the presence of a disease condition or a susceptibility to a disease condition, wherein said condition is associated with an over- or under-expression of, or activity of, a polypeptide of the invention, comprising contacting a cell, tissue or cell extract of said patient with an antibody which is specific for a polypeptide of the invention, and detecting the amount or activity of said polypeptide.

The invention also relates to a method to determine the presence of a disease condition or susceptibility to a disease condition, wherein said condition is associated with a mutated nicotinic alpha-7 receptor, comprising identifying such a mutation in a nicotinic alpha-7 receptor isolated from a patient.

In another aspect, the invention relates to methods to screen for agents that modulate (e.g., stimulate or inhibit) expression or activity of a polypeptide of the invention, or of a polynucleotide which encodes it, comprising contacting a cell, preferably from neuronal tissue, or a tissue cell extract with a putative modulatory agent, and measuring the amount or activity of said polypeptide or polynucleotide, including, e.g., monitoring net charge flow through the alpha-7 receptor/channel in response to said agent, measuring the total amount of charge flowing across the membrane of said cell, or measuring the change in a calcium-sensitive dye present in said cell, in response to said agent.

The invention also relates to methods to screen for agents which bind to a polypeptide or polynucleotide of the invention, comprising contacting an inventive polypeptide or polynucleotide with a putative binding agent and determining the presence of a bound complex (e.g., a nucleic acid hybrid, antigen-antibody complex, protein-protein interaction, ligand-target complex, or the like). Methods of the invention can be performed in vitro, ex vivo, or in vivo.

In another aspect, the invention relates to a transgenic animal (e.g., a mouse), preferably a non-human mammal, comprising at least one copy of an alpha-7 receptor polynucleotide of the invention, wherein the animal overexpresses functional alpha-7 receptor, or a functional fragment or analog thereof, compared to a non-transgenic animal. In another aspect, the invention relates to a knockout animal, e.g., a primate, whose genome lacks a gene expressing a functional alpha-7 receptor or functional fragment or variant thereof; or to a transgenic animal in which the natural alpha-7 receptor is replaced by a heterologous transgenic. Such transgenic animals can have a modified response to a nicotinic receptor ligand, e.g., altered desensitization properties.

In another aspect, the invention relates to a pharmaceutical composition comprising a polypeptide or polynucleotide of the invention and a pharmaceutically acceptable carrier. In another aspect, the invention relates to a prophylactic or therapeutic method of treating a disease condition mediated by, or associated with, aberrant expression and/or activity of the alpha-7 receptor, comprising administering to a patient in need thereof an agent which modulates the expression and/or activity of said alpha-7 receptor.

Polypeptides

A polypeptide of the present invention may be a recombinant polypeptide, a natural polypeptide or a synthetic or semi-synthetic polypeptide, or combinations thereof, preferably a recombinant polypeptide. As used herein, the terms polypeptide, oligopeptide and protein are interchangeable. A nicotinic alpha-7 receptor channel polypeptide of the present invention can comprise various domains, including, e.g., a signal sequence at about amino acid positions 1-22, and at least four transmembrane domains: M1 at about amino acid positions 231-255, M2 at about amino acid positions 262-283, M3 at about amino acid positions 293-315, and M4 at about amino acid positions 470-494. This numbering is in accordance with the amino acid sequence set forth in FIG. 2 (SEQ ID NO:2).

The polypeptides of the present invention are preferably provided in an isolated form, and may be purified, e.g. to homogeneity. The term “isolated,” when referring, e.g., to a polypeptide or polynucleotide, means that the material is removed from its original environment (e.g., the natural environment if it is naturally occurring), and isolated or separated from at least one other component with which it is naturally associated. For example, a naturally-occurring polypeptide present in its natural living host is not isolated, but the same polypeptide, separated from some or all of the coexisting materials in the natural system, is isolated. Such polypeptides could be part of a composition, and still be isolated in that such composition is not part of its natural environment.

The terms “fragment” or “variant,” when referring to a polypeptide of the invention, mean a polypeptide which retains substantially at least one of the biological functions or activities of the polypeptide. Such a biological function or activity can be, e.g., any of those described above, and includes having the ability to react with an antibody, i.e., having a epitope-bearing peptide. Fragments or variants of the polypeptide of FIGS. 2, 4, or 6 (SEQ ID NOS: 2, 4, or 6) have sufficient similarity to FIG. 2, 4, or 6 (SEQ ID NOS: 2, 4, or 6) so that at least one activity (e.g., an activity expressed by a functional domain thereof, or the ability to react with an antibody or antigen-binding fragment of the invention) of the native polypeptides is retained. Biological functions or activities of alpha-7 nicotinic acetylcholine receptor polypeptide, which forms a homo-pentameric channel in vitro or when expressed in cells, include nicotinic ligand-binding and ligand-gated ion conductance, e.g., the channel is selectively permeable to Ca²⁺ and other cations, when activated by a receptor agonist. Polypeptide fragments of the invention may be of any size that is compatible with the objects of the invention. They may range in size from the smallest specific epitope (e.g., about 6 amino acids) to a nearly full-length gene product (e.g., a single amino acid shorter than the polypeptides sequences in FIG. 2, 4, or 6 (SEQ ID NOS: 2, 4, or 6). A preferred fragment that would still retain biological function excludes the signal peptide of the sequence (for example, signal sequence may include amino acids 1-22 of FIG. 2, 4, or 6 (SEQ ID NOS: 2, 4, or 6)).

Fragments of the polypeptides of the present invention may be employed, e.g., for producing the corresponding fill-length polypeptide by peptide synthesis, e.g., as intermediates for producing the fill-length polypeptides; for inducing the production of antibodies or antigen-binding fragments; as “query sequences” for the probing of public databases, or the like.

A variant of a polypeptide of the invention may be, e.g., (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the polypeptide is fused with another compound, such as a compound to increase the half-life of the polypeptide (for example, polyethylene glycol), or (iv) one in which additional amino acids are fused to the polypeptide, such as a leader or secretory sequence or a sequence which is employed for purification of the polypeptide, commonly for the purpose of creating a genetically engineered form of the protein that is susceptible to secretion from a cell, such as a transformed cell. The additional amino acids may be from a heterologous source, or may be endogenous to the natural gene.

Variant polypeptides belonging to type (i) above include, e.g., mutants, analogs and derivatives. A variant polypeptide can differ in amino acid sequence by, e.g., one or more additions, substitutions, deletions, insertions, inversions, fusions, and truncations or a combination of any of these.

In one embodiment, the monkey nicotinic alpha-7 acetylcholine receptor subunit comprises at least one mutation. Preferably, the mutation is in the M2 domain of the subunit. The mutation can result in slower desensitization than in the wild type nicotinic alpha-7 polypeptide, enhancing Ca⁺⁺ assay read-outs. Desensitization can be determined routinely, e.g., using the methods described in the examples, or in the publications cited herein. M2 domains (generally about 20 amino acids in length) of the alpha-7 polypeptides have been characterized from a variety of species and have been shown to play an important role in ion permeation through nicotinic alpha-7 receptor channels. See, e.g., Changeux et al. (1992) Q. Rev. Biophys. 25, 395-432; Bertrand et al. (1993) Proc. Natl. Acad. Sci. 90, 6971-6975; and Revah et al. (1991) Nature 353, 846-849. Examples of mutations in the M2 domain include, for example, Leu at position 270 substituted with Thr (L270T) and Val at position 274 substituted with Thr (V274T). Other mutants include those single and double monkey nicotinic alpha-7 mutations equivalent to the single and double chicken nicotinic alpha-7 mutations L247T, L247S, L247F, L247V, V251T, T244Q, E237A/V251T, E237A/L247T, E237A/L247S, E237A/L247V, E237A/L247F, E237A/T244Q, E237A/L254T, or E237A/L255T. The equivalent single and double monkey nicotinic alpha-7 mutations are, respectively, L270T, L270S, L270F, L270V, V274T, T267Q, E260A/V274T, E260A/L270T, E260A/L270S, E260A/L270V, E260A/L270F, E260A/T267Q, E260A/L277T, or E260A/L278T. In addition, other residues, when mutated, may also lead to slower desensitization, e.g., residues that face the channel lumen and are aligned along the meridian of an alpha-helix. See, e.g., Bertrand et al. (1995) The Neurosciences 7, 75-90 and Bertrand et al. (1993), Current Opinion in Cell Biology 5, 688-693.

In addition to mutations in the M2 region, mutations can be made in other regions or domains of the polypeptide, and the corresponding polynucleotide sequences which encode it. This includes one or more mutations, such as: A65V, RI56W, S193N, K208R, K208S, M395V, A398V and/or A398T. These mutations are preferably made in combination with mutations in the M2 region, and therefore include double-mutants, triple-mutants, etc. FIGS. 5 and 6 (SEQ ID NOS: 5 and 6) show an example of a double-mutant comprising amino acid substitutions in the M2 region (L270T) and in an other region (S193N).

Variant polypeptides belonging to type (ii) above include, e.g., modified polypeptides. Known polypeptide modifications include, but are not limited to, glycosylation, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formatin, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well-known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in many basic texts, such as Proteins—Structure and Molecular Properties, 2nd ed., T. E. Creighton, W. H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al. (1990) Meth. Enzymol. 182:626-646 and Rattan et al. (1992) Ann. N.Y. Acad. Sci. 663:48-62.

Variant polypeptides belonging to type (iii) are well-known in the art and include, e.g., PEGulation or other chemical modifications.

Variants polypeptides belonging to type (iv) above include, e.g., preproteins or proproteins which can be activated by cleavage of the proprotein portion to produce an active mature polypeptide. Variants include a variety of hybrid, chimeric or fusion polypeptides. Typical example of such variants are discussed elsewhere herein.

Many other types of variants are known to those of skill in the art. For example, as is well known, polypeptides are not always entirely linear. For instance, polypeptides may be branched as a result of ubiquitination, and they may be circular, with or without branching, generally as a result of post-translation events, including natural processing events and events brought about by human manipulation which do not occur naturally. Circular, branched and branched circular polypeptides may be synthesized by non-translational natural processes and by synthetic methods.

Modifications or variations can occur anywhere in a polypeptide, including the peptide backbone, the amino acid side-chains and the amino or carboxyl termini. The same type of modification may be present in the same or varying degree at several sites in a given polypeptide. Also, a given polypeptide may contain more than one type of modification. Blockage of the amino or carboxyl group in a polypeptide, or both, by a covalent modification, is common in naturally-occurring and synthetic polypeptides. For instance, the amino-terminal residue of polypeptides made in E. coli, prior to proteolytic processing, is often N-formylmethionine. The modifications can be a function of how the protein is made. For recombinant polypeptides, for example, the modifications are determined by the host cell posttranslational modification capacity and the modification signals in the polypeptide amino acid sequence. Accordingly, when glycosylation is desired, a polypeptide can be expressed in a glycosylating host, generally a eukaryotic cell. Insect cells often carry out the same posttranslational glycosylations as mammalian cells and, for this reason, insect cell expression systems have been developed to efficiently express mammalian proteins having native patterns of glycosylation. Similar considerations apply to other modifications.

Variant polypeptides can be fully functional or can lack function in one or more activities, e.g., in any of the functions or activities described above. Among the many types of useful variations are, e.g., those that exhibit alteration of receptor activity. For example, one embodiment involves a variation at the binding site that results in binding of a ligand, but without receptor activity. A further useful variation at the same site can result in altered affinity for a particular ligand of the receptor. Another useful variation provides a fusion protein in which one or more domains or subregions are operationally fused to one or more domains or subregions from another nicotinic alpha-7 isoform or family member.

As noted above, the polypeptides of the present invention include, e.g., isolated polypeptides comprising the sequence of FIG. 2, 4, or 6 (SEQ ID NOS: 2, 4, or 6) (in particular the mature polypeptides) and fragments thereof. The polypeptides of the invention also include polypeptides, which have varying degrees of sequence homology (identity) thereto. The invention also encompasses polypeptides having a lower degree of sequence identity, but having sufficient similarity so as to perform one or more of the functions or activities exhibited by the rhesus monkey nicotinic alpha-7 receptor.

In accordance with the present invention, the term “percent identity” or “percent identical,” when referring to a sequence, means that a sequence is compared to a claimed or described sequence after alignment of the sequence to be compared (the “Compared Sequence”) with the described or claimed sequence (the “Reference Sequence”). The Percent Identity is then determined according to the following formula:
Percent Identity=100[1−(C/R)]
wherein C is the number of differences between the Reference Sequence and the Compared Sequence over the length of alignment between the Reference Sequence and the Compared Sequence wherein (i) each base or amino acid in the Reference Sequence that does not have a corresponding aligned base or amino acid in the Compared Sequence and (ii) each gap in the Reference Sequence and (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the hereinabove calculated Percent Identity is less than the specified Percent Identity.

In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence.

The description herein for percent identity or percent homology is intended to apply equally to nucleotide or amino acid sequences.

The comparison of sequences and determination of percent identity and similarity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

A preferred, non-limiting example of such a mathematical algorithm is described in Karlin et al. (1993) Proc. Natl. Acad. Sci. USA 90:5873-5877. Such an algorithm is incorporated into the NBLAST and XBLAST programs (version 2.0) as described in Altschul et al. (1997) Nucleic Acids Res. 25:3389-3402. When utilizing BLAST and Gapped BLAST programs, the default parameters of the respective programs (e.g., NBLASST) can be used. In one embodiment, parameters for sequence comparison can be set at score=100, wordlength-12, or can be varied (e.g., W=5 or W=20).

In a preferred embodiment, the percent identity between two amino acid sequences is determined using the Needleman et al. (1970) (J. Mol. Biol. 48:444-453) algorithm which has been incorporated into the GAP program in the GCG software package using either a BLOSUM 62 matrix or a PAM250 matrix, and a gap weight of 16, 14, 12, 10, 8, 6, or 4 and a length weight of 1,2,3,4,5 or 6. In yet another preferred embodiment, the percent identity between two nucleotide sequences is determined using the GAP program I the GCG software package (Devereux et al. (1984) Nucleic Acids Res. 12 (1):387) using a NWSgapdna. CMP matrix and a gap weight of 40, 50, 60, 70, or 80 and a length weight of 1,2,3,4,5 or 6.

Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the CGC sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis et al. (1994) Comput. Appl. Biosci. 10:3-5; and FASTA described in Pearson et al. (1988) PNAS 85:2444-8.

In accordance with the present invention, the term “substantially homologous,” when referring to a protein sequence, means that the amino acid sequences are at least about 90-95%, preferably 97-99% or more identical.

Conditions of “high stringency,” as used herein, means, for example, incubating a blot overnight (e.g., at least 12 hours) with a long polynucleotide probe in a hybridization solution containing, e.g., about 5×SSC, 0.5% SDS, 100 μg/ml denatured salmon sperm DNA and 50% formamide, at 42° C. Blots can be washed at high stringency conditions that allow, e.g., for less than 5% bp mismatch (e.g., wash twice in 0.1×SSC and 0.1% SDS for 30 min at 65° C.), thereby selecting sequences having, e.g., 95% or greater sequence identity.

Other non-limiting examples of high stringency conditions include a final wash at 65° C. in aqueous buffer containing 30 mM NaCl and 0.5% SDS. Another example of high stringent conditions is hybridization in 7% SDS, 0.5 M NaPO₄, pH 7, 1 mM EDTA at 50° C., e.g., overnight, followed by one or more washes with a 1% SDS solution at 42° C. Whereas high stringency washes can allow for less than 5% mismatch, reduced or low stringency conditions can permit up to 20% nucleotide mismatch. Hybridization at low stringency can be accomplished as above, but using lower formamide conditions, lower temperatures and/or lower salt concentrations, as well as longer periods of incubation time.

Polypeptides, and fragments or variants thereof, within the present invention may also contain unbroken stretches of amino acids, e.g., about 6, 8, 10, 12, 14, 15, 20, 25, 30, 35, 40, 50, 60, 70, 80 or 90 amino acids. A further preferred fragment excludes the signal sequence (for example, signal sequence may include amino acids 1-22 of FIG. 2, 4, or 6 (SEQ ID NOS: 2, 4, or 6).

As used with respect to the polypeptides (and polynucleotides) of the present invention, the term fragment refers to a sequence that is a subset of a larger sequence (i.e., a continuous or unbroken sequence of residues within a larger sequence). Peptides already present in the art are, of course, excluded.

The polypeptides, and fragments thereof, of the present invention may be found in the cells and tissues of any species of animal, but are preferably found in cells from mammals, e.g., mouse, rat, rabbit, farm animals, pets, etc., especially the cells of primates. In any given animal, the polypeptides and fragments thereof within the present invention may be found in a variety of tissues. Methods of determining the tissue or cellular location of such polypeptides are conventional and include, e.g., conventional methods of immunohistochemistry. Various alpha-7 receptors are found in, e.g., many brain regions, hippocampus, thalamus, cerebellum, and hypothalamus. (Quik, M., J. of Comparative Neurology (2000) 425, 58-69.)

In particular, the alpha-7 receptor polypeptides and fragments thereof of the invention are found in cells, tissues and organs of the nervous system, most especially the brain, for example in the various regions of the hippocampus.

Nucleic Acids

As discussed above, the invention includes, e.g., cDNA (SEQ ID NOs: 1 or 3) encoding full length polypeptides of the invention, and fragments thereof.

The polynucleotides of SEQ FIG. 1, 3, or 5 (SEQ ID NOS: 1, 3, or 5) contain open reading frames available for the coding of polypeptide amino acid sequences. For the sequence of SEQ ID NO: 1, the open reading frame (or ORF) coding for the polypeptide of SEQ ID NO: 2 is found at nucleotides 7-1515 (with nucleotides 1513-1515 representing the “TAA” termination codon). For the sequence of SEQ ID NO: 3, the open reading frame (or ORF) coding for the polypeptide of SEQ ID NO. 4 is found at nucleotides 7-1515 (with nucleotides 1513-1515 representing the “TAA” termination codon). For the sequence of SEQ ID NO: 5, the open reading frame (or ORF) coding for the polypeptide of SEQ ID NO: 6 is found at nucleotide 7-1515 with nucleotides 1513-1515 representing the “TAA” termination codon).

As used herein, the phrase “an isolated polynucleotide which is SEQ ID NO,” or “an isolated polynucleotide which is selected from SEQ ID NO,” refers to an isolated nucleic acid molecule from which the recited sequence was obtained (i.e., the mRNA). Because of sequencing errors, typographical errors, etc., the actual naturally-occurring sequence may differ from a SEQ ID listed herein. Thus, the phrase indicates the specific molecule from which the sequence was derived, rather than a molecule having that exact recited nucleotide sequence, analogously to how a culture depository number refers to a specific cloned fragment in a cryotube.

A polynucleotide of the present invention may be a recombinant polynucleotide, a natural polynucleotide, or a synthetic or semi-synthetic polynucleotide, or combinations thereof. As used herein, the terms polynucleotide, oligonucleotide, oligomer and nucleic acid are interchangeable.

As used herein, the term “gene” means a segment of DNA involved in producing a polypeptide chain; it may include regions preceding and following the coding region (leader and trailer) as well as intervening sequences (introns) between individual coding segments (exons). cDNAs lack the corresponding introns. The invention includes isolated genes (e.g., genomic clones) which encode polypeptides of the invention.

Polynucleotides of the invention may be RNA, PNA, or DNA, e.g., cDNA, genomic DNA, and synthetic or semi-synthetic DNA, or combinations thereof. The DNA may be triplex, double-stranded or single-stranded, and if single stranded, may be the coding strand or non-coding (anti-sense) strand. It can comprise hairpins or other secondary structures. The RNA includes oligomers (including those having sense or antisense strands), mRNAs (e.g., having the alternative splices of the alpha-7 receptor), polyadenylated RNA, total RNA, single strand or double strand RNA, or the like. DNA/RNA duplexes are also encompassed by the invention.

The polynucleotides and fragments thereof of the present invention may be of any size that is compatible with the objects of the invention, e.g., of any desired size that is effective to achieve a desired specificity when used as a probe. Polynucleotides may range in size, e.g., from the smallest specific probe (e.g., about 10-12 nucleotides ) to greater than a full-length cDNA, e.g., in the case of a fusion polynucleotide or a polynucleotide that is part of a genomic sequence; fragments may be as large as, e.g., one nucleotide shorter than a full-length cDNA.

A fragment of a polynucleotide according to the invention may be used, e.g., as a hybridization probe, as discussed elsewhere herein.

Many types of variants of polynucleotides are encompassed by the invention including, e.g., (i) one in which one or more of the nucleotides is substituted with another nucleotide, or which is otherwise mutated; or (ii) one in which one or more of the nucleotides is modified, e.g., includes a subtituent group; or (iii) one in which the polynucleotide is fused with another compound, such as a compound to increase the half-life of the polynucleotide; or (iv) one in which additional nucleotides are covalently bound to the polynucleotide, such a sequences encoding a leader or secretory sequence or a sequence which is employed for purification of the polypeptide. The additional nucleotides may be from a heterologous source, or may be endogenous to the natural gene.

Polynucleotide variants belonging to type (i) above include, e.g., polymorphisms, including single nucleotide polymorphisms (SNPs), and mutants. Variant polynucleotides can comprise, e.g., one or more additions, insertions, deletions, substitutions, transitions, transversions, inversions, chromosomal translocations, variants resulting from alternative splicing events, or the like, or any combinations thereof

A coding sequence which encodes a polypeptide (e.g., a mature polypeptide) of the invention may be identical to the coding sequence shown in FIG. 1, 3, or 5 (SEQ ID NOS: 1, 3, or 5) or a fragment thereof, or may be a different coding sequence, which coding sequence, as a result of the redundancy or degeneracy of the genetic code, encodes the same polypeptide as the DNA of FIG. 1, 3, or 5 (SEQ ID NOS: 1, 3, or 5) or a fragment thereof Such a peptide is sometimes referred to herein as a “degenerate variant.” Alternatively, the coding sequence may encode a polypeptide that is substantially homologous to the polypeptide of FIG. 2, 4, or 6 (SEQ ID NOS: 2, 4, or 6) or a fragment thereof

A polynucleotide of the invention may have a coding sequence which is a naturally or non-naturally occurring allelic variant of a coding sequence encompassed by the sequence in SEQ ID NO: 1. As known in the art, an allelic variant is an alternate form of a polynucleotide sequence which may have a substitution, deletion or addition of one or more nucleotides, which in general does not substantially alter the function of the encoded polypeptide.

Other variant sequences, located in a coding sequence or in a regulatory sequence, may affect (enhance or decrease) the production of, or the function or activity of, a polypeptide of the invention.

Polynucleotide variants belonging to type (ii) above include, e.g., modifications such as the attachment of detectable markers (avidin, biotin, radioactive elements, fluorescent tags and dyes, energy transfer labels, energy-emitting labels, binding partners, etc.) or moieties which improve expression, uptake, cataloging, tagging, hybridization, detection, and/or stability. The polynucleotides can also be attached to solid supports, e.g., nitrocellulose, magnetic or paramagnetic microspheres (e.g., as described in U.S. Pat. Nos. 5,411,863; 5,543,289; for instance, comprising ferromagnetic, supermagnetic, paramagnetic, superparamagnetic, iron oxide and polysaccharide), nylon, agarose, diazotized cellulose, latex solid microspheres, polyacrylamides, etc., according to a desired method. See, e.g., U.S. Pat. Nos. 5,470,967; 5,476,925; 5,478,893.

Polynucleotide variants belonging to type (iii) above are well known in the art and include, e.g., various lengths of polyA⁺ tail, 5′ cap structures, and nucleotide analogs, e.g., inosine, thionucleotides, or the like.

Polynucleotide variants belonging to type (iv) above include, e.g., a variety of chimeric, hybrid or fusion polynucleotides. For example. a polynucleotide of the invention can comprise a coding sequence and additional non-naturally occurring or heterologous coding sequence (e.g., sequences coding for leader, signal, secretory, targeting, enzymatic, fluorescent, antibiotic resistance, and other functional or diagnostic peptides); or a coding sequence and non-coding sequences, e.g., untranslated sequences at either a 5′ or 3′ end, or dispersed in the coding sequence, e.g., introns.

More specifically, the present invention includes polynucleotides wherein the coding sequence for the polypeptide (e.g., a mature polypeptide) is fused in the same reading frame to a polynucleotide sequence (e.g., a heterologous sequence), e.g. one which aids in expression and secretion of a polypeptide from a host cell, for example, a leader sequence which functions as a secretory sequence for controlling transport of a polypeptide from the cell and/or a transmembrane anchor which facilitates attachment of the polypeptide to a cellular membrane. A polypeptide having a leader sequence is a preprotein and may have the leader sequence cleaved by the host cell to form a mature form of the polypeptide. The polynucleotides may also encode for a proprotein which is the mature protein plus additional N-terminal amino acid residues. A mature protein having a prosequence is a proprotein and is generally an inactive form of the protein. Once the prosequence is cleaved an active protein remains.

Polynucleotides of the present invention may also have a coding sequence fused in frame to a marker sequence that allows for identification and/or purification of the polypeptide of the present invention. The marker sequence may be, e.g., a hexa-histidine tag (e.g., as supplied by a pQE-9 vector) to provide for purification of the mature polypeptide fused to the marker in the case of a bacterial host, or, for example, the marker sequence may be a hemagglutinin (HA) tag when a mammalian host, e.g. COS-7 cells, is used. The HA tag corresponds to an epitope derived from the influenza hemagglutinin protein (Wilson, I., et al., Cell, 37:767 (1984)).

Other types of polynucleotide variants will be evident to one of skill in the art. For example, the nucleotides of a polynucleotide can be joined via various known linkages, e.g., ester, sulfamate, sulfamide, phosphorothioate, phosphoramidate, methylphosphonate, carbamate, etc., depending on the desired purpose, e.g., resistance to nucleases, such as RNAse H, improved in vivo stability, etc. See, e.g., U.S. Pat. No. 5,378,825. Any desired nucleotide or nucleotide analog can be incorporated, e.g., 6-mercaptoguanine, 8-oxo-guanine, etc. Also, polynucleotides of the invention may have a coding sequence derived from another genetic locus of an organism, providing it has a substantial homology to, e.g., part or all of the sequence of FIG. 1, 3, or 5 (SEQ ID NOS: 1, 3, or 5) or from another organism (e.g., an ortholog). It is understood that variants exclude any sequences disclosed prior to the invention.

Polynucleotides according to the present invention can be labeled according to any desired method. The polynucleotide can be labeled using radioactive tracers such as, e.g., ³²P, ³⁵S, ³H, or ¹⁴C. The radioactive labeling can be carried out according to any method, such as, for example, terminal labeling at the 3′ or 5′ end using a radiolabeled nucleotide, polynucleotide kinase (with or without dephosphorylation with a phosphatase) or a ligase (depending on the end to be labeled). A non-radioactive labeling can also be used, combining a polynucleotide of the present invention with residues having immunological properties (antigens, haptens), a specific affinity for certain reagents (ligands), properties enabling detectable enzyme reactions to be completed (enzymes or coenzymes, enzyme substrates, or other substances involved in an enzymatic reaction), or characteristic physical properties, such as fluorescence or the emission or absorption of light at a desired wavelength, etc.

The present invention includes polynucleotides encoding all of the polypeptides and fragments or variants thereof, as disclosed hereinabove. For example, a polynucleotide of the invention may comprise a sequence which has a sequence identity of at least about 65-100%, (e.g., at least about 70-75%, 80-85%, 90-95% or 97-99%) to, or which is substantially homologous to, or which hybridizes under conditions of high stringency to, the nucleotide sequence of SEQ ID NO: 1, or to a fragment thereof; or which is complementary to one of those sequences.

The term “substantially homologous,” when referring to polynucleotide sequences, means that the nucleotide sequences are at least about 90-95% or 97-99% or more identical.

Constructs

The present invention also relates to recombinant constructs that contain vectors plus polynucleotides of the present invention. Such constructs comprise a vector, such as a plasmid or viral vector, into which a polynucleotide sequence of the invention has been inserted, in a forward or reverse orientation.

Large numbers of suitable vectors are known to those of skill in the art, and many are commercially available. The following vectors are provided by way of example; Bacterial: pQE70, pQE60, pQE-9 (Qiagen), pBS, pD10, phagescript, psiX174, pBluescript SK, pBSKS, pNH8A, pNH16a, pNH18A, pNH46A (Stratagene); pTRC99a, pKK223-3, pKK233-3, pDR540, pRIT5 (Pharmacia); Eukaryotic: pWLNEO, pSV2CAT, pOG44, pXT1, pSG (Stratagene) pSVK3, pBPV, pMSG, pSVL (Pharmacia). However, any other plasmid or vector may be used as long as it is replicable and viable in the host.

In a preferred embodiment, the vector is an expression vector, into which a polynucleotide sequence of the invention is inserted so as to be operatively linked to an appropriate expression control (regulatory) sequence(s) (e.g., promoters and/or enhancers) which directs mRNA synthesis. Appropriate expression control sequences, e.g., regulatable promoter or regulatory sequences known to control expression of genes in prokaryotic or eukaryotic cells or their viruses, can be selected for expression in prokaryotes (e.g., bacteria), yeast, plants, mammalian cells or other cells. Preferred expression control sequences are derived from highly-expressed genes, e.g., from operons encoding glycolytic enzymes such as 3-phosphoglycerate kinase (PGK), α-factor, acid phosphatase, or heat shock proteins, among others. Such expression control sequences can be selected from any desired gene, e.g using CAT (chloramphenicol transferase) vectors or other vectors with selectable markers. Two appropriate vectors for such selection are pKK232-8 and pCM7.

Particular named bacterial promoters which can be used include lacI, lacZ, T3, T7, gpt, lambda P_{R}, P_{L} and trp. Eukaryotic promoters include CMV immediate early, HSV thymidine kinase, early and late SV40, adenovirus promoters, LTRs from retrovirus, and mouse metallothionein-I. Selection of the appropriate vector and promoter is well within the level of ordinary skill in the art.

Transcription of the DNA encoding the polypeptides of the present invention by higher eukaryotes can be increased by inserting an enhancer sequence into the expression vector. Enhancers are cis-acting elements of DNA, usually about from 10 to 300 bp that act on a promoter to increase its transcription. Representative examples include the SV40 enhancer on the late side of the replication origin bp 100 to 270, a cytomegalovirus early promoter enhancer, the polyoma enhancer on the late side of the replication origin, and adenovirus enhancers.

Generally, recombinant expression vectors also include origins of replication. An expression vector may contain a ribosome binding site for translation initiation, a transcription termination sequence, a polyadenylation site, splice donor and acceptor sites, and/or 5′ flanking or non-transcribed sequences. DNA sequences derived from the SV40 splice and polyadenylation sites may be used to provide required nontranscribed genetic elements. The vector may also include appropriate sequences for amplifying expression. In addition, expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli.

Large numbers of suitable expression vectors are known to those of skill in the art, and many are commercially available. Suitable vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, adeno-associated virus, TMV, fowl pox virus, and pseudorabies. However, any other vector may be used as long as it is replicable and viable in a host. Appropriate cloning and expression vectors for use with prokaryotic and eukaryotic hosts are described, e.g., by Sambrook, et al., Molecular Cloning: A Laboratory Manual, Second Edition, Cold Spring Harbor, N.Y., (1989), Wu et al, Methods in Gene Biotechnology (CRC Press, New York, N.Y., 1997), Recombinant Gene Expression Protocols, in Methods in Molecular Biology, Vol. 62, (Tuan, ed., Humana Press, Totowa, N.J., 1997), and Current Protocols in Molecular Biology, (Ausabel et al, Eds.,), John Wiley & Sons, NY (1994-1999).

In a preferred embodiment, a Baculovirus-based expression system is used. Baculoviruses represent a large family of DNA viruses that infect mostly insects. The prototype is the nuclear polyhedrosis virus (AcMNPV) from Autographa californica, which infects a number of lepidopteran species. One advantage of the baculovirus system is that recombinant baculoviruses can be produced in vivo. Following co-transfection with transfer plasmid, most progeny tend to be wild type and a good deal of the subsequent processing involves screening. To help identify plaques, special systems are available that utilize deletion mutants. By way of non-limiting example, a recombinant AcMNPV derivative (called BacPAK6) has been reported in the literature that includes target sites for the restriction nuclease Bsu36I upstream of the polyhedrin gene (and within ORF 1629) that encodes a capsid gene (essential for virus viability). Bsf36I does not cut elsewhere in the genome and digestion of the BacPAK6 deletes a portion of the ORF1629, thereby rendering the virus non-viable. Thus, with a protocol involving a system like Bsu361-cut BacPAK6 DNA most of the progeny are non-viable so that the only progeny obtained after co-transfection of transfer plasmid and digested BacPAK6 is the recombinant because the transfer plasmid, containing the exogenous DNA, is inserted at the Bsu36I site thereby rendering the recombinants resistant to the enzyme. See Kitts and Possee, A method for producing baculovirus expression vectors at high frequency, BioTechniques, 14, 810-817 (1993). For general procedures, see King and Possee, The Baculovirus Expression System: A Laboratory Guide, Chapman and Hall, New York (1992) and Recombinant Gene Expression Protocols, in Methods in Molecular Biology, Vol. 62, (Tuan, ed., Humana Press, Totowa, N.J., 1997), at Chapter 19, pp. 235-246.

Appropriate DNA sequences may be inserted into a vector by any of a variety of procedures. In general, the DNA sequence is inserted into an appropriate restriction endonuclease site(s) by procedures known in the art. Such procedures and others are deemed to be within the scope of those skilled in the art. Conventional procedures for this and other molecular biology techniques discussed herein are found in many readily available sources, e.g., Sambrook, et al., Molecular Cloning: A Laboratory Manual, Second Edition, Cold Spring Harbor, N.Y., (1989). If desired, a heterologous structural sequence is assembled in an expression vector in appropriate phase with translation initiation and termination sequences, and preferably, a leader sequence capable of directing secretion of translated protein into the periplasmic space or extracellular medium.

Transformed Cells and Methods of Producing Polypeptides of the Invention

The present invention also relates to host cells which are transformed/transfected/transduced (i.e, wherein a polynucleotide of the present invention has been introduced into the host cell, and is therefore not the endogenous, naturally-occurring gene which is normally present in the cell) with constructs such as those described above, and to progeny of said cells, especially where such cells result in a stable cell line that can be used for assays of nicotinic alpha-7 receptor activity, e.g., in order to identify agents which modulate nicotinic alpha-7 receptor activity, and/or for production (e.g., preparative production) of the polypeptides of the invention. The host cell can contain a vector comprising a polynucleotide operably linked to an expression control sequence (e.g., a promoter) which enables expression of the polypeptide. Once expressed in the cell, the alpha-7 nicotinic polypeptides can assemble with each other to form a functional homo-pentameric channel which is located on the surface of the cell (i.e., on the surface cell membrane). As representative examples of appropriate hosts, there may be mentioned: bacterial cells, such as E. coli, Streptomyces, Salmonella typhimurium; fungal cells, such as yeast; insect cells such as Drosophila S2 and Spodoptera Sf9 (and other insect expression systems); animal cells, including mammalian cells such as CHO, COS (e.g., the COS-7 lines of monkey kidney fibroblasts described by Gluzman, Cell, 23:175 (1981)), C127, 3T3, CHO, HeLa, BHK or Bowes melanoma cell lines; muscle cells; neuronal cells; brain cells; oocytes (such as from Xenopus or other frog species), QT6 (ATCC CRL-1708), QM7 (ATCC CRL-1962), plant cells, etc. The selection of an appropriate host is deemed to be within the knowledge of those skilled in the art based on the teachings herein.

In another embodiment, the host cells are insect cells of Spodoptera species, most especially SF9 cells, from Spodoptera frugiperda. Polypeptides (e.g., full length polypeptides) of the present invention are readily obtainable from insect cells using a baculovirus expression vector. Such expression is readily characterized using methods well known in the art.

Introduction of a construct into a host cell can be effected by, e.g., calcium phosphate transfection, DEAE-Dextran mediated transfection, lipofection a gene gun, or electroporation (Davis, L., Dibner, M., Battey, I., Basic Methods in Molecular Biology, (1986)).

Following transformation of a suitable host strain and growth of the host strain to an appropriate cell density, the selected promoter can be induced by appropriate means (e.g., temperature shift or chemical induction) if desired, and cells cultured for an additional period. The engineered host cells are cultured in conventional nutrient media modified as appropriate for activating promoters (if desired), selecting transformants or amplifying the genes of the present invention. The culture conditions, such as temperature, pH and the like, are those previously used with the host cell selected for expression, and will be apparent to the ordinarily skilled artisan.

Cells are typically harvested by centrifugation, disrupted by physical or chemical means, and the resulting crude extract retained for further purification. Alternatively, when a heterologous polypeptide is secreted from the host cell into the culture fluid, supernatants of the culture fluid can be used as a source of the protein. Microbial cells employed in expression of proteins can be disrupted by any convenient method, including freeze-thaw cycling, sonication, mechanical disruption, or use of cell lysing agents, such methods being well known to those skilled in the art.

The polypeptide can be recovered and purified from recombinant cell cultures by conventional methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography, or the like. Protein refolding steps can be used, as necessary, in completing configuration of the mature protein. High performance liquid chromatography (HPLC) can be employed for final purification steps.

In addition to the methods described above for producing polypeptides recombinantly from a prokaryotic or eukaryotic host, polypeptides of the invention can be prepared from natural sources, or can be prepared by chemical synthetic procedures (e.g., synthetic or semi-synthetic), e.g., with conventional peptide synthesizers. Cell-free translation systems can also be employed to produce such proteins using RNAs derived from the DNA constructs of the present invention. Proteins of the invention can also be expressed in, and isolated and/or purified from, transgenic animals or plants. Procedures to make and use such transgenic organisms are conventional in the art. Some such procedures are described elsewhere herein.

Antibodies, Antigen-binding Fragments or Other Specific Binding Partners

The polypeptides, their fragments or variants thereof, or cells expressing them can also be used as immunogens to produce specific antibodies, or antigen-binding fragments, thereto. By a “specific” antibody or antigen-binding fragment is meant one which binds selectively (preferentially) to nicotinic alpha-7 receptor of the invention, or to a fragment or variant thereof. An antibody “specific” for a polypeptide means that the antibody recognizes a defined sequence of amino acids within or including the polypeptide. Preferred antibodies are those that recognize epitopes which are specific for the monkey alpha-7 nicotinic acetylcholine receptor of the present invention, e.g., regions which differ in sequence identity between the corresponding protein expressed in other species.

Antibodies of the invention can be, for example, polyclonal or monoclonal antibodies. The present invention also includes chimeric, recombinant, single chain, and partially or fully humanized antibodies, as well as Fab fragments, or the product of a Fab expression library, and fragments thereof. The antibodies can be IgM, IgG, subtypes, IgG2A, JgG1, etc. Various procedures known in the art may be used for the production of such antibodies and fragments.

Antibodies generated against the polypeptides corresponding to a sequence of the present invention can be obtained, e.g., by direct injection of the polypeptides into an animal or by administering the polypeptides to an animal, e.g., goat, rabbit, mouse, chicken, etc., preferably a non-human. The antibody so obtained will then bind the polypeptide itself. In this manner, even a sequence encoding only a fragment of the polypeptides can be used to generate antibodies binding the whole native polypeptides. Such antibodies can then be used to isolate the polypeptide from tissue expressing that polypeptide. Antibodies can also be generated by administering naked DNA. See, e.g., U.S. Pat. Nos. 5,703,055; 5,589,466; and 5,580,859.

For preparation of monoclonal antibodies, any technique which provides antibodies produced by continuous cell line cultures can be used. Examples include, e.g., the hybridoma technique (Kohler and Milstein, 1975, Nature, 256:495-497), the trioma technique, the human B-cell hybridoma technique (Kozbor et al., 1983, Immunology Today 4:72), and the EBV-hybridoma technique to produce human monoclonal antibodies (Cole, et al., 1985, in Monoclonal Antibodies and Cancer Therapy, Alan R. Liss, Inc., pp. 77-96).

Techniques described for the production of single chain antibodies (e.g., U.S. Pat. No. 4,946,778) can be adapted to produce single chain antibodies to immunogenic polypeptide products of this invention. Also, transgenic animals may be used to express partially or fully humanized antibodies to immunogenic polypeptide products of this invention.

The invention also relates to other specific binding partners, which include, e.g., aptamers and PNA.

Transgenic and Knockout Animals

The invention disclosed herein also relates to a non-human transgenic animal comprising within its genome one or more copies of the polynucleotides encoding the novel polypeptides of the invention. The transgenic animals of the invention may contain within their genome multiple copies of the polynucleotides encoding the polypeptides of the invention, or one copy of a gene encoding such polypeptide but wherein said gene is linked to a promoter (e.g., a regulatable promoter) that will direct expression (preferably overexpression) of said polypeptide within some, or all, of the cells of said transgenic animal. In a preferred embodiment, expression of a polypeptide of the invention occurs preferentially in brain tissue, e.g., hippocampus. A variety of non-human transgenic organisms are encompassed by the invention, including e.g., drosophila, C.elegans, zebrafish and yeast. The transgenic animal of the invention is preferably a mammal, e.g., a cow, goat, sheep, rabbit, non-human primate, monkey, rhesus monkey, rat, or mouse.

Methods of producing transgenic animals are well within the skill of those in the art, and include, e.g., homologous recombination, mutagenesis (e.g., ENU, Rathkolb et al., Exp. Physiol., 85(6):635-644, 2000), and the tetracycline-regulated gene expression system (e.g., U.S. Pat. No. 6,242,667), and will not be described in detail herein. [See e.g., Wu et al, Methods in Gene Biotechnology, CRC 1997, pp. 339-366; Jacenko, O., Strategies in Generating Transgenic Animals, in Recombinant Gene Expression Protocols, Vol. 62 of Methods in Molecular Biology, Humana Press, 1997, pp 399-424]

Transgenic organisms are useful, e.g. for providing a source of a polynucleotide or polypeptide of the invention, or for identifying and/or characterizing agents that modulate expression and/or activity of such a polynucleotide or polypeptide. Transgenic animals are also useful as models for disease conditions related to, e.g., overexpression of a polynucleotide or polypeptide of the invention.

The present invention also relates to a non-human knockout animal whose genome lacks or fails to express a functional nicotinic alpha-7 receptor or functional analog thereof (i.e., the gene is functionally disrupted), such animal commonly being referred to as a “knockout” animal, especially a “knock-out mouse.”

Functional disruption of the gene can be accomplished in any effective way, including, e.g., introduction of a stop codon into any part of the coding sequence such that the resulting polypeptide is biologically inactive (e.g., because it lacks a catalytic domain, a ligand binding domain, etc.), introduction of a mutation into a promoter or other regulatory sequence that is effective to turn it off, or reduce transcription of the gene, insertion of an exogenous sequence into the gene which inactivates it (e.g., which disrupts the production of a biologically-active polypeptide or which disrupts the promoter or other transcriptional machinery), deletion of sequences from the nicotinic alpha-7 receptor gene, etc. Examples of transgenic animals having functionally disrupted genes are well known, e.g., as described in U.S. Pat. Nos. 6,239,326, 6,225,525, 6,207,878, 6,194,633, 6,187,992, 6,180,849, 6,177,610, 6,100,445, 6,087,555, 6,080,910, 6,069,297, 6,060,642, 6,028,244, 6,013,858, 5,981,830, 5,866,760, 5,859,314, 5,850,004, 5,817,912, 5,789,654, 5,777,195, and 5,569,824. Knock-outs can be homozygous or heterozygous.

For creating functional disrupted genes, and other gene mutations, homologous recombination technology is of special interest since it allows specific regions of the genome to be targeted. Using homologous recombination methods, genes can be specifically inactivated, specific mutations can be introduced, and exogenous sequences can be introduced at specific sites. These methods are well known in the art, e.g., as described in the patents above. See, also, Robertson, Biol. Reproduc., 44(2):238-245, 1991. Generally, the genetic engineering is performed in an embryonic stem (ES) cell, or other pluripotent cell line (e.g., adult stem cells, EG cells), and that genetically-modified cell (or nucleus) is used to create a whole organism. Nuclear transfer can be used in combination with homologous recombination technologies.

For example, nicotinic alpha-7 locus can be disrupted in monkey ES cells using a positive-negative selection method (e.g., Mansour et al., Nature, 336:348-352, 1988). In this method, a targeting vector can be constructed which comprises a part of the gene to be targeted. A selectable marker, such as neomycin resistance genes, can be inserted into an alpha-7 exon present in the targeting vector, disrupting it. When the vector recombines with the ES cell genome, it disrupts the function of the gene. The presence in the cell of the vector can be determined by expression of neomycin resistance. See, e.g., U.S. Pat. No. 6,239,326. Cells having at least one functionally disrupted gene can be used to make chimeric and germline animals, e.g., animals having somatic and/or germ cells comprising the engineered gene. Homozygous knock-out animals can be obtained from breeding heterozygous knock-out animals. See, e.g., U.S. Pat. No. 6,225,525.

The present invention also relates to a transgenic non-human animal whose genome comprises one or more genes coding for monkey alpha-7 receptor disclosed herein in place of the mammalian gene otherwise coding for said non-human isoform.

A knock-out animal, or animal cell, lacking one or more fictional nicotinic alpha-7 receptor genes can be useful in a variety of applications, including as an animal model for an nicotinic alpha-7 receptor-mediated or related condition, for drug screening assays, as a source of tissues deficient in nicotinic alpha-7 receptor activity, as the starting material for generating an animal in which the endogenous nicotinic alpha-7 receptor is replaced with monkey alpha-7 receptor, and any of the utilities mentioned in any issued U.S. Patent on transgenic animals, including, U.S. Pat. Nos. 6,239,326, 6,225,525, 6,207,878, 6,194,633, 6,187,992, 6,180,849, 6,177,610, 6,100,445, 6,087,555, 6,080,910, 6,069,297, 6,060,642, 6,028,244, 6,013,858, 5,981,830, 5,866,760, 5,859,314, 5,850,004, 5,817,912, 5,789,654, 5,777,195, and 5,569,824. For instance, nicotinic alpha-7 receptor deficient animal cells can be utilized to study activities related to, e.g., neuronal diseases, such as Alzheimer's disease and schizophrenia. By knocking-out nicotinic alpha-7 receptors e.g., one at a time, the physiological pathways using nicotinic alpha-7 receptors can be dissected out and identified.

In addition to the methods mentioned above, transgenic or knock-out animals can be prepared according to known methods, including, e.g., by pronuclear injection of recombinant genes into pronuclei of 1-cell embryos, incorporating an artificial yeast chromosome into embryonic stem cells, gene targeting methods, embryonic stem cell methodology, cloning methods, nuclear transfer methods. See, also, e.g., U.S. Pat. Nos. 4,736,866; 4,873,191; 4,873,316; 5,082,779; 5,304,489; 5,174,986; 5,175,384; 5,175,385; 5,221,778; Gordon et al., Proc. Natl. Acad. Sci., 77:7380-7384, 1980; Palmiter et al., Cell, 41:343-345, 1985; Palmiter et al., Ann. Rev. Genet., 20:465-499, 1986; Askew et al., Mol. Cell. Bio., 13:4115-4124, 1993; Games et al. Nature, 373:523-527, 1995; Valancius and Smithies, Mol. Cell. Bio., 11:1402-1408, 1991; Stacey et al., Mol. Cell. Bio., 14:1009-1016, 1994; Hasty et al., Nature, 350:243-246, 1995; Rubinstein et al., Nucl. Acid Res., 21:2613-2617,1993; Cibelli et al., Science, 280:1256-1258, 1998. For guidance on recombinase excision systems, see, e.g., U.S. Pat. Nos. 5,626,159, 5,527,695, and 5,434,066. See also, Orban, P. C., et al., “Tissue- and Site-Specific DNA Recombination in Transgenic Mice,” Proc. Natl. Acad. Sci. USA, 89:6861-6865 (1992); O'Gorman, S., et al., “Recombinase-Mediated Gene Activation and Site-Specific Integration in Mammalian Cells,” Science, 251:1351-1355 (1991); Sauer, B., et al., “Cre-stimulated recombination at loxP-Containing DNA sequences placed into the mammalian genome,” Polynucleotides Research, 17(1):147-161 (1989); Gagneten, S. et al. (1997) Nucl. Acids Res. 25:3326-3331; Xiao and Weaver (1997) Nucl. Acids Res. 25:2985-2991; Agah, R. et al. (1997) J. Clin. Invest. 100:169-179; Barlow, C. et al. (1997) Nucl. Acids Res. 25:2543-2545; Araki, K. et al. (1997) Nucl. Acids Res. 25:868-872; Mortensen, R. N. et al. (1992) Mol. Cell. Biol. 12:2391-2395 (G418 escalation method); Lakhlani, P. P. et al. (1997) Proc. Natl. Acad. Sci. USA 94:9950-9955 (“hit and run”); Westphal and Leder (1997) Curr. Biol. 7:530-533 (transposon-generated “knock-out” and “knock-in”); Templeton, N. S. et al. (1997) Gene Ther. 4:700-709 (methods for efficient gene targeting, allowing for a high frequency of homologous recombination events, e.g., without selectable markers); PCT International Publication WO 93/22443 (functionally-disrupted).

A polynucleotide according to the present invention can be introduced into any non-human animal, including a non-human mammal, mouse (Hogan et al., Manipulating the Mouse Embryo: A Laboratory Manual, Cold Spring Harbor Laboratory, Cold Spring Harbor, N.Y., 1986), pig (Hammer et al., Nature, 315:343-345, 1985), sheep (Hammer et al., Nature, 315:343-345, 1985), cattle, rat, or primate. See also, e.g., Church, 1987, Trends in Biotech. 5:13-19; Clark et al., Trends in Biotech. 5:20-24, 1987); and DePamphilis et al., BioTechniques, 6:662-680, 1988. Transgenic animals can be produced by the methods described in U.S. Pat. No. 5,994,618, and utilized for any of the utilities described therein.

Conditions Related to Nicotinic Alpha-7 Receptor Expression

The alpha-7 receptor of the instant invention is involved in a variety of functions and activities, e.g. as discussed elsewhere hereinabove, and aberrant expression and/or activity of nicotinic alpha-7 receptor is associated with a variety of disease conditions. This invention relates, e.g., to the detection (e.g., determination of the presence or absence) and/or quantitation of polypeptides or polynucleotides of the invention that are related to such conditions; and to the diagnosis and/or prevention, treatment, or amelioration of symptoms, of such alpha-7 receptor-mediated or nicotinic alpha-7 receptor-related conditions. The invention also relates to methods of identifying agents that modulate (i.e., increase or decrease) the expression and/or activity of polypeptides or polynucleotides associated with such conditions, and to methods of identifying polypeptide or polynucleotide alterations or mutants that are associated with such conditions.

Methods of the invention relate to the treatment and/or prophylaxis of various diseases and conditions, particularly psychotic diseases, neurodegenerative diseases involving a dysfunction of the cholinergic system, and conditions of memory and/or cognition impairment, including, for example, schizophrenia, anxiety, mania, depression, manic depression [examples of psychotic disorders], Tourette's syndrome, Parkinson's disease, Huntington's disease [examples of neurodegenerative diseases], cognitive disorders (such as Alzheimer's disease, Lewy Body Dementia, Amyotrophic Lateral Sclerosis, memory impairment, memory loss, cognition deficit, attention deficit, Attention Deficit Hyperactivity Disorder), and other uses such as treatment of nicotine addiction, inducing smoking cessation, treating pain (i.e., analgesic use), providing neuroprotection, and treating jetlag. See, e.g., WO 97/30998; WO 99/03850; WO 00/42044; WO 01/36417; Holladay et al., J.Med. Chem., 40:26, 4169-94 (1997); Schmitt et al., Annual Reports Med. Chem., Chapter 5, 41-51 (2000); Stevens et al., Psychopharmatology, (1998) 136: 320-27 (1998); and Shytle et al., Molecular Psychiatry, (2002), 7, pp. 525-535.

In one embodiment, methods of the invention relate to psychotic diseases, neurodegenerative diseases involving a dysfunction of the cholinergic system, and conditions of memory and/or cognition impairment, including, for example, schizophrenia, anxiety, mania, depression, manic depression [examples of psychotic disorders], Tourette's syndrome, Parkinson's disease, Huntington's disease [examples of neurodegenerative diseases], and/or cognitive disorders (such as Alzheimer's disease, Lewy Body Dementia, Amyotrophic Lateral Sclerosis, memory impairment, memory loss, cognition deficit, attention deficit, and Attention Deficit Hyperactivity Disorder).

Neurodegenerative disorders included within the methods of the present invention include, but are not limited to, treatment and/or prophylaxis of Alzheimer's diseases, Pick's disease, diffuse Lewy Body disease, progressive supranuclear palsy (Steel-Richardson syndrome), multisystem degeneration (Shy-Drager syndrome), motor neuron diseases including amyotrophic lateral sclerosis, degenerative ataxias, cortical basal degeneration, ALS-Parkinson's-Dementia complex of Guam, subacute sclerosing panencephalitis, Huntington's disease, Parkinson's disease, synucleinopathies, primary progressive aphasia, striatonigral degeneration, Machado-Joseph disease/spinocerebellar ataxia type 3, olivopontocerebellar degenerations, Gilles De La Tourette's disease, bulbar, pseudobulbar palsy, spinal muscular atrophy, spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, prion diseases (such as Creutzfeldt-Jakob, Gerstmann-Sträussler-Scheinker disease, Kuru and fatal familial insomnia), and neurodegenerative disorders resulting from cerebral ischemia or infarction including embolic occlusion and thrombotic occlusion as well as intracranial hemorrhage of any type (including, but not limited to, epidural, subdural, subarachnoid and intracerebral), and intracranial and intravertebral lesions (including, but not limited to, contusion, penetration, shear, compression and laceration).

The present invention further relates to memory impairment due to, for example, mild cognitive impairment due to aging, Alzheimer's disease, schizophrenia, Parkinson's disease, Huntington's disease, Pick's disease, Creutzfeld-Jakob disease, depression, aging, head trauma, stroke, CNS hypoxia, cerebral senility, multiinfaret dementia and other neurological conditions, as well as HIV and cardiovascular diseases.

In addition, the methods of the invention relate to age-related dementia and other dementias and conditions with memory loss including age-related memory loss, senility, vascular dementia, diffuse white matter disease (Binswanger's disease), dementia of endocrine or metabolic origin, dementia of head trauma and diffuse brain damage, dementia pugilistica and frontal lobe dementia. See, e.g., WO 99/62505.

Amyloid precursor protein (APP) and Aβ peptides derived therefrom, e.g., Aβ₁₋₄₀, Aβ₁₋₄₂, and other fragments, are known to be involved in the pathology of Alzhemier's disease. The Aβ₁₋₄₂ peptides are not only implicated in neurotoxicity but also are known to inhibit cholinergic transmitter function. Further, it has been determined that Aβ peptides bind to α-7 nAChRs. Thus, agents which block the binding of the Aβ peptides to α-7 nAChRs are useful for treating neurodegenerative diseases. See, e.g., WO 99/62505. In addition, stimulation α-7 nAChRs can protect neurons against cytotoxicity associated with Aβ peptides. See, e.g., Kihara, T. et al., Ann. Neurol., 1997, 42, 159.

Thus, in accordance with an embodiment of the invention, the methods are related to treatment or prevention of dementia in an Alzheimer's patient, which comprises administering to the subject a therapeutically effective amount of a compound able to inhibit the binding of an amyloid beta peptide (preferably, Aβ₁₋₄₂) with nAChRs, preferable α-7 nAChRs.

The present invention also provides methods related to treating other amyloidosis diseases, for example, hereditary cerebral angiopathy, nonneuropathic hereditary amyloid, Down's syndrome, macroglobulinemia, secondary familial Mediterranean fever, Muckle-Wells syndrome, multiple myeloma, pancreatic- and cardiac-related amyloidosis, chronic hemodialysis anthropathy, and Finnish and Iowa amyloidosis.

In addition, nicotinic receptors have been implicated as playing a role in the body's response to alcohol ingestion. Thus, agonists for α-7nAChR's can be used in the treatment of alcohol withdrawal and in anti-intoxication therapy. Thus, in accordance with an embodiment of the invention, the methods are related to treating a patient for alcohol withdrawal or treating a patient with anti-intoxication therapy.

Agonists for the α-7nAChR subtypes can also be used for neuroprotection against damage associated with strokes and ischemia and glutamate-induced excitotoxicity. Thus, in accordance with an embodiment of the invention there is provided a method related to providing for neuroprotection against damage associated with strokes and ischemia and glutamate-induced excitotoxicity.

As noted above, agonists for the α-7nAChR subtypes can also be used in the treatment of nicotine addiction, inducing smoking cessation, treating pain, and treating jetlag. Thus, in accordance with an embodiment of the invention there is provided a method related to treatment of suffering from nicotine addiction, pain, and/or jetlag, or a method of inducing smoking cessation.

The condition of memory impairment is manifested by impairment of the ability to learn new information and/or the inability to recall previously learned information. Memory impairment is a primary symptom of dementia and can also be a symptom associated with such diseases as Alzheimer's disease, schizophrenia, Parkinson's disease, Huntington's disease, Pick's disease, Creutzfeld-Jakob disease, HIV, cardiovascular disease, and head trauma as well as age-related cognitive decline.

Thus, in accordance with an embodiment of the invention there is provided a method related to treating a patient suffering from, for example, mild cognitive impairment (MCI), vascular dementia (VaD), age-associated cognitive decline (AACD), amnesia associated w/open-heart-surgery, cardiac arrest, and/or general anesthesia, sleep deprivation induced cognitive impairment, chronic fatigue syndrome, narcolepsy, AIDS-related dementia, epilepsy-related cognitive impairment, Down's syndrome, Alcoholism related dementia, drug/substance induced memory impairments, Dementia Puglistica (Boxer Syndrome),and animal dementia (e.g. dogs, cats, horses, etc. ).

Screening Assays for Modulatory Agents and Assays for the Determination of Nicotinic Alpha-7 Receptor Levels and/or Activities

This invention provides methods of screening agents, in vitro or in vivo (e.g., in cell-based assays or in animal models), to identify those agents that modulate (e.g., enhance, stimulate, restore, inhibit, block, stabilize, destabilize, increase, facilitate, up-regulate, activate, amplify, augment, induce, decrease, down-regulate, diminish, lessen, reduce, etc.) synthesis and/or activity of the nicotinic alpha-7 receptors of the invention to a putative agent, in the presence or absence of an nicotinic alpha-7 stimulatory agent, and measuring the activity of the alpha-7, e.g., as indicated by Ca⁺⁺ influx, compared to the activity in the absence of the putative agent,measuring the total amount of charge flowing across the membrane of said cell, or measuring the change in a calcium-sensitive dye present in said cell, in response to said agent.

As used herein, a compound that “modulates the activity of a neuronal nicotinic AchR” refers to a compound that alters the activity of nicotinic alpha-7 receptor so that activity of the nicotinic alpha-7 receptor is different in the presence of the compound than in the absence of the compound. In particular, such compounds include agonists or antagonists. The term agonist refers to a substance, such as acetylcholine, that activates receptor function; and the term antagonist refers to a substance that interferes with receptor function. Typically, the effect of an antagonist is observed as a blocking of activation by an agonist. Antagonists include competitive and non-competitive antagonists. A competitive antagonist (or competitive blocker) interacts with or near the site specific for the agonist (e.g., ligand or neurotransmitter) for the same or closely situated site. A non-competitive antagonist or blocker inactivates the functioning of receptor by interacting with a site other than the site that interacts with the agonist.

A “nicotinic cholinergic agonist” is a compound that binds to and activates a nicotinic acetylcholine receptor. By “activates” is intended the elicitation of one or more pharmacological, physiological, or electrophysiological response. Such a response includes, but is not limited to, cell membrane depolarization and increased permeability to Ca⁺⁺ and other cations. Agents can also be partial agonists, e.g., when an agent is only partly effective as an agonist.

A “nicotinic cholinergic antagonist” is a substance that binds to a nicotinic acetylcholine receptor and prevents agonists from activating the receptor. Pure antagonists do not activate the receptor, but some substances may have mixed agonist and antagonist properties. Nicotinic cholinergic channel blockers block the ability of agonists to elicit current flow through the nicotinic acetylcholine receptor channel, but do so by blocking the channel rather than by preventing agonists from binding to and activating the receptor.

A “nicotinic cholinergic receptor” intends a substance that influences the activity of the nicotinic acetylcholine receptor through interaction at one or more sites other than the classic agonist binding site. The regulator may itself increase or decrease receptor activity, or may influence agonist activity (for example, potentiating responses) without itself eliciting an overt change in channel current. A single substance can have different properties at different nicotinic acetylcholine receptor subtypes, for example, being an agonist at one receptor and antagonist at another, or an antagonist at one and a channel blocker at another.

By “nAChr modulator” is intended a substance that may act as an agonist, antagonist, channel blocker, or regulator.

As understood by those of skill in the art, assay methods for identifying compounds that modulate rhesus monkey neuronal nicotinic alpha-7 activity (e.g., agonists and antagonists) generally require comparison to a control. One type of “control” cell or “control” culture is a cell or culture that is treated substantially the same as the cell or culture exposed to the test compound, except the control culture is not exposed to test compound.

In another embodiment, this invention provides methods of screening agents, in vitro or in vivo (e.g., in cell-based assays or in animal models), to identify those agents that modulate (e.g., enhance, stimulate, restore, inhibit, block, stabilize, destabilize, increase, facilitate, up-regulate, activate, amplify, augment, induce, decrease, down-regulate, diminish, lessen, reduce, etc.) expression of the nicotinic alpha-7 receptors of the invention.

In a further embodiment, this invention provides methods of screening agents, in vitro or in vivo (e.g., in cell-based assays or in animal models), to identify those agents that modulate (e.g., enhance, stimulate, restore, inhibit, block, stabilize, destabilize, increase, facilitate, up-regulate, activate, amplify, augment, induce, decrease, down-regulate, diminish, lessen, reduce, etc.) the transport of an alpha-7 subunit, or of a receptor comprising at least one such alpha-7 subunit, to the cell membrane.

Assays for Ca⁺⁺ uptake can be performed either in the absence of a ligand or following stimulation by an appropriate ligand. Appropriate stimulatory ligands include, e.g., nicotine or nicotinic acid (preferably the (−) enantiomer), carbamyl choline, cytosine, acetylcholine, epibatidine, or alpha-7 specific ligands such as GTS-21, 4OH-GTS-21. Non alpha-7 specific ligands stimulation can be shown by the reduction or blocking of the signal with conventional inhibitors such as, e.g., methyllycaconitine or alpha-bungarotoxin. Calcium uptake can be measured conventionally, e.g., using calcium-sensitive dyes, such as Fluo-3AM, Fluo-3, Fluo-4, Fluo-4AM, Rhod-2, Calcium Green-1. Calcium Green-2, etc. See, also the examples below.

Among the types of modulatory agents that can be tested and identified by the methods of the invention are, e.g., small chemical compounds (e.g., inorganic or organic molecules, such as conventional combinatorial libraries), polypeptides, peptides, or peptide analogs, polynucleotides, antibodies that bind specifically to the polypeptides of the invention, or the like.

Without wishing to be bound to any particular mechanism, it is proposed that an inhibitory or stimulatory agent may act on the ligand binding moiety of the alpha-7 receptor, on an allosteric binding moiety, or on an element of the ion channel, thereby modulating activity of the protein; or the agent may enter cells and, e.g., bind directly to the DNA neighboring the sequences coding for the polypeptides of the invention, thereby increasing or decreasing their expression; or the agent may enter the cell and affect post-transcriptional processing, thereby modulating the protein activity; or the agent may affect the transport of the alpha-7 to the cell membrane.

Any of the assays described herein can, of course, be adapted to any of a variety of high throughput methodologies, as can the generation, identification and characterization of putative inhibitory or stimulatory agents. Agents identified on the basis of their ability to modulate alpha-7 receptor expression or activity may also be used for modulating other neuronal nicotinic acetylcholine receptors, and/or for diagnosing or treating disease conditions related thereto.

Antisense Oligonucleotides and Ribozymes

Potential antagonists or inhibitors of the invention include isolated antisense oligonucleotides, or antisense constructs which express antisense oligonucleotides, both of which classes of molecules can be prepared using conventional technology. Antisense technology can be used to control gene expression through methods based on binding of a polynucleotide to DNA or RNA. Without wishing to be bound to any particular mechanism, types of antisense oligonucleotides and proposed mechanisms by which they function include, e.g., the following: The 5′ coding portion of a polynucleotide sequence which encodes for a mature polypeptide of the present invention can be used to design an antisense oligonucleotide (e.g., an RNA, DNA, PNA etc. oligonucleotide) of any site which is compatible with the objects of the invention, e.g., of from about 10 to 40 base pairs in length. The antisense oligonucleotide can hybridize to the mRNA and block translation of the mRNA molecule into an alpha-7 receptor polypeptide (see e.g., Okano, J. Neurochem., 56:560 (1991); Oligodeoxynucleotides as Antisense Inhibitors of Gene Expression, CRC Press, Boca Raton, Fla. (1988)). Alternatively, an oligonucleotide can be designed to be complementary to a region of the gene involved in transcription (see, e.g, Lee et al., Nucl. Acids Res., 6:3073 (1979); Cooney et al, Science, 241:456 (1988); and Dervan et al., Science, 251: 1360 (1991)), thereby preventing transcription and the production of alpha-7 receptors. For further guidance on administering and designing antisense, see, e.g., U.S. Pat. Nos. 6,200,960, 6,200,807, 6,197,584, 6,190,869, 6,190,661, 6,187,587, 6,168,950, 6,153,595, 6,150,162, 6,133,246, 6,117,847, 6,096,722, 6,087,343, 6,040,296, 6,005,095, 5,998,383, 5,994,230, 5,891,725, 5,885,970, and 5,840,708.

Antisense polynucleotides can comprise modified, nonnaturally-occurring nucleotides and linkages between the nucleotides (e.g., modification of the phosphate-sugar backbone; methyl phosphonate, phosphorothioate, or phosphorodithioate linkages; and 2′-O-methyl ribose sugar units), e.g., to enhance in vivo or in vitro stability, to confer nuclease resistance, to modulate uptake, to modulate cellular distribution and compartmentalization, etc. Any effective nucleotide or modification can be used, including those already mentioned, as known in the art, etc., e.g., disclosed in U.S. Pat. Nos. 6,133,438; 6,127,533; 6,124,445; 6,121,437; 5,218,103 (e.g., nucleoside thiophosphoramidites); 4,973,679; Sproat et al., “2′-O-Methyloligoribonucleotides: synthesis and applications,” Oligonucleotides and Analogs A Practical Approach, Eckstein (ed.), IRL Press, Oxford, 1991, 49-86; Iribarren et al., “2′-O-Alkyl Oligoribonucleotides as Antisense Probes,” Proc. Natl. Acad. Sci. USA, 1990, 87, 7747-7751; Cotton et al., “2′-O-methyl, 2′-O-ethyl oligoribonucleotides and phosphorothioate oligodeoxyribonucleotides as inhibitors of the in vitro U7 snRNP-dependent mRNA processing event,” Nucl. Acids Res., 1991, 19, 2629-2635. Effective amounts of antisense oligonucleotides as described above can be administered to a patient in need thereof by conventional means.

Antisense oligonucleotides can also be delivered to cells via, e.g., plasmids or other vectors, wherein the antisense sequence is operably linked to an expression control sequence. In this manner, RNA or DNA antisense is expressed in a cell and inhibits production of alpha-7 receptors. A total length of about 36 nucleotides can be used in cell culture with cationic lipisomes to facilitate cellular uptake, but for in vivo use, preferably shorter oligonucleotides are administered, e.g., about 25 nucleotides.

In another embodiment, ribozymes corresponding to specific sequences can be introduced into cells such that they cleave nicotinic alpha-7 receptor coding or regulatory sequences. Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. Ribozyme molecules designed to catalytically cleave target gene mRNA transcripts can also be used to prevent translation of target gene mRNA and expression of target gene. (See, e.g., PCT International Publication WO90/11364, published Oct. 4, 1990; Sarver et al., 1990, Science 247:1222-1225). While ribozymes that cleave mRNA at site specific recognition sequences can be used to destroy target gene mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591. Preferably the ribozyme is engineered so that the cleavage recognition site is located near the 5′ end of the target mRNA, i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO 88/04300 by University Patents Inc.; Been and Cech, 1986, Cell, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in target gene.

As in the antisense approach, the ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target gene messages and inhibit translation. Because ribozymes, unlike antisense molecules, are catalytic, a lower intracellular concentration is required for efficiency.

Diagnostics/Assays for Alpha-7 Receptor Polypeptides

The present invention provides for a means of diagnosing or staging actual or potential disease conditions involving altered levels of nicotinic alpha-7 receptors by determining the amounts (e.g., the presence or absence, or the quantity) of the polypeptides of the invention, or their levels of activity, in an animal suspected of having such a disease condition or being at risk therefor. For example, the invention provides a process for diagnosing a disease in an animal afflicted therewith, or diagnosing a susceptibility to a disease in an animal at risk thereof, wherein said disease is related, for example, to an over- or under-expression or activity of av alpha-7 receptor according to the invention, comprising determining the amount of said alpha-7 receptor or the level of said alpha-7 receptor activity in a cell from said animal, wherein said animal is preferably a mammal, most preferably a primate.

When assaying samples for diagnostic purposes, using any of the methods described herein, samples may be obtained from any suitable cell, tissue, organ, or bodily fluid from a patient, including but not limited to blood, urine, saliva, tissue biopsy and autopsy material. In one embodiment, samples for diagnosis are taken from cells or tissues in which high levels of alpha-7 receptor expression are normally observed, e.g., neurological tissue. In a preferred embodiment, the disease conditions to be diagnosed involve loss of memory as a primary or secondary effect thereof, especially loss of long term memory, and the cells tested are typically neurons, especially those of the brain, for example, neurons of the hippocampal region (e.g., in hippocampal slices).

Enzymatic assays for the various activities exhibited by nicotinic alpha-7 receptors are conventional. Some such assays are described above. Detection and/or quantitation of protein levels can be accomplished by any of a variety of conventional methods, e.g., methods based on antibodies or antigen-specific fragments of the invention. Immunological assays include, e.g., ELISA, RIA and FACS assays. A two-site, monoclonal-based immunoassay, utilizing antibodies reactive to two non-interfering epitopes on a alpha-7 receptor polypeptide are preferred, but a competitive binding assay may be employed. These and other assays are described, e.g., in Hampton et al. (1990). Serological Methods, a Laboratory Manual, APS Press, St. Paul, Minn.

The invention provides methods for diagnosing a disease or susceptibility thereto wherein said disease is related to production of an aberrant form of an alpha-7 receptor according to the invention, e.g., one resulting from a genetic mutation. Such aberrant (or variant) proteins include those described above, e.g., proteins having amino acid substitutions, deletions, inversions, insertions, rearrangements (e.g., as a result of aberrant splicing events) or inappropriate post-translational modifications. Aberrant proteins may exhibit increased or decreased activity of any of the functions described elsewhere herein. Aberrant proteins may also exhibit increased or decreased interactions with other proteins, such as, e.g.,protein kinases, cytoskeletal proteins, etc.

Variant proteins (e.g., mutants or muteins) can be detected by any of a variety of conventional methods. For example, antibodies or antigen binding fragments can be used to detect the presence of aberrant forms of the polypeptides disclosed herein, using immunological methods such as those described above.

In accordance with the present invention, an antibody or antigen-binding fragment can be present in a kit, where the kit includes, e.g., one or more antibodies or antigen-binding fragments, a desired buffer, detection compositions, proteins (e.g., wild type) to be used as controls, etc.

Diagnostic/Assays for Alpha-7 Receptor Nucleic Acid

Assays involving polynucleotides can be used to determine the presence or absence of a nucleic acid in a sample and/or to quantify it, or to detect a mutation or polymorphism. Such assays can be used, e.g., for diagnostic, prognostic, research, or forensic purposes. The assays can be, e.g., membrane-based, solution-based, or chip-based. Assays can be performed at the single-cell level, or in a sample comprising many cells, where the assay is “averaging” expression over the entire collection of cells and tissue present in the sample.

Any suitable assay format can be used, including, but not limited to, Southern blot analysis, Northern blot analysis, polymerase chain reaction (“PCR”) (e.g., Saiki et al., Science, 241:53, 1988; U.S. Pat. Nos. 4,683,195, 4,683,202, and 6,040,166; PCR Protocols: A Guide to Methods and Applications, Innis et al., eds., Academic Press, New York, 1990), reverse transcriptase polymerase chain reaction (“RT-PCR”), anchored PCR, rapid amplification of cDNA ends (“RACE”) (e.g., Schaefer in Gene Cloning and Analysis: Current Innovations, Pages 99-115, 1997), ligase chain reaction (“LCR”) (EP 320 308), one-sided PCR (Ohara et al., Proc. Natl. Acad. Sci., 86:5673-5677, 1989), indexing methods (e.g., U.S. Pat. No. 5,508,169), in situ hybridization, differential display (e.g., Liang et al., Nucl. Acid. Res., 21:3269-3275, 1993; U.S. Pat. Nos. 5,262,311, 5,599,672 and 5,965,409; WO97/18454; Prashar and Weissman, Proc. Natl. Acad. Sci., 93:659-663, and U.S. Pat. Nos. 6,010,850 and 5,712,126; Welsh et al., Nucleic Acid Res., 20:4965-4970, 1992, and U.S. Pat. No. 5,487,985) and other RNA fingerprinting techniques, nucleic acid sequence based amplification (“NASBA”) and other transcription based amplification systems (e.g., U.S. Pat. Nos. 5,409,818 and 5,554,527; WO 88/10315), polynucleotide arrays (e.g., U.S. Pat. Nos. 5,143,854, 5,424,186; 5,700,637, 5,874,219, and 6,054,270; PCT WO 92/10092; PCT WO 90/15070), QBeta Replicase (PCT/US87/00880), Strand Displacement Amplification (“SDA”), Repair Chain Reaction (“RCR”), nuclease protection assays, subtraction-based methods, Rapid-Scan™, etc. Additional useful methods include, but are not limited to, e.g., template-based amplification methods, competitive PCR (e.g., U.S. Pat. No. 5,747,251), redox-based assays (e.g., U.S. Pat. No. 5,871,918), Taqman-based assays (e.g., Holland et al., Proc. Natl. Acad, Sci., 88:7276-7280, 1991; U.S. Pat. Nos. 5,210,015 and 5,994,063), real-time fluorescence-based monitoring (e.g., U.S. Pat. 5,928,907), molecular energy transfer labels (e.g., U.S. Pat. Nos. 5,348,853, 5,532,129, 5,565,322, 6,030,787, and 6,117,635; Tyagi and Kramer, Nature Biotech., 14:303-309, 1996). Any method suitable for single cell analysis of gene or protein expression can be used, including in situ hybridization, immunocytochemistry, MACS, FACS, flow cytometry, etc. For single cell assays, expression products can be measured using antibodies, PCR, or other types of nucleic acid amplification (e.g, Brady et al., Methods Mol. & Cell. Biol. 2, 17-25, 1990; Eberwine et al., 1992, Proc. Natl. Acad. Sci., 89, 3010-3014, 1992; U.S. Pat. No. 5,723,290). These and other methods can be carried out conventionally, e.g., as described in the mentioned publications.

The invention provides methods for diagnosing a disease in an animal afflicted therewith, or diagnosing susceptibility to a disease in an animal at risk thereof, wherein said disease is related, for example, to an over- or under-expression of a polynucleotide encoding an alpha-7 receptor according to the invention, comprising determining the amount of said polynucleotide in a cell from said animal, wherein said animal is preferably a mammal and most preferably a primate. Any of the assay methods described herein, or otherwise known in the art, can be used to determine the presence of and/or to quantitate, such polynucleotides.

Furthermore, detection of a mutated or polymorphic form of a gene allows a diagnosis of a disease or a susceptibility to a disease, which results from expression of a mutated alpha-7 receptor polypeptide that may have, for example, increased or decreased activity in certain tissues. Such mutations include, e.g., any of those described elsewhere herein, e.g., point mutations, insertions, deletions, substitutions, transversions, and chromosomal translocations.

Individuals carrying mutations in a gene of the present invention may be detected at the DNA level by a variety of techniques. Genomic DNA may be used directly for detection or may be amplified enzymatically by using PCR (Saiki et al., Nature, 324:163-166 (1986); Innis et al eds., (1996) PCR Protocols: A Guide to Methods in Amplification, Academic Press, New York) prior to analysis. RNA or cDNA may also be used for the same purpose. As an example, PCR primers complementary to the nucleic acid encoding an alpha-7 receptor can be used to identify and analyze mutations. For example, deletions and insertions can be detected by a change in size of the amplified product in comparison to the normal genotype. Point mutations can be identified, e.g., by hybridizing amplified DNA to radiolabeled RNA or radiolabeled antisense DNA sequences. Perfectly matched sequences can be distinguished from mismatched duplexes by a variety of methods, including, e.g., RNase A digestion or by differences in melting temperatures. Rapid sequencing methods can be employed.

Sequence differences between the reference gene and genes having mutations may be revealed by the direct DNA sequencing method. In addition, cloned DNA segments may be employed as probes to detect specific DNA segments. The sensitivity of this method is greatly enhanced when combined with PCR. For example, a sequencing primer is used with double-stranded PCR product or a single-stranded template molecule generated by a modified PCR. The sequence determination is performed by conventional procedures with radiolabeled nucleotide or by automatic sequencing procedures with fluorescent-tags.

A polynucleotide sequence coding for part or all of an alpha-7 receptor of the invention may act as a reference for the development of probes, e.g., as long as 30 to 45 nucleotides, or longer, that can be used to probe the genome of animals suspected of being at risk for disease, or having such disease. Probes corresponding to regulatory sequences e.g., sequences which govern the amount of mRNA coding for the alpha-7 receptor of the invention, or of the alpha-7 receptor protein produced, can also be used. Such regulatory sequences include, e.g., promoter or enhancer elements, sequences which govern splicing events, stability of nucleic acid or protein, termination/polyadenylation and/or intracellular localization of mRNAs or proteins.

Genetic testing based on DNA sequence differences may be achieved by detection of alteration in electrophoretic mobility of DNA fragments in gels with or without denaturing agents. Small sequence deletions and insertions can be visualized by high resolution gel electrophoresis. DNA fragments of different sequences may be distinguished on denaturing formamide gradient gels in which the mobilities of different DNA fragments are retarded in the gel at different positions according to their specific melting or partial melting temperatures (see, e.g., Myers et al., Science, 230:1242 (1985)), or by mass spectroscopy analysis.

In addition, sequence changes at specific locations may also be revealed by nuclease protection assays, such as RNase and S1 protection or the chemical cleavage method (e.g, Cotton et al., PNAS, USA, 85:4397-4401 (1985)) and these are deemed within the methods of the invention.

Thus, the detection of a specific DNA sequence may be achieved by methods such as, e.g, hybridization, RNase protection, chemical cleavage, direct DNA sequencing or the use of restriction enzymes, (e.g., Restriction Fragment Length Polymorphisms (RFLP)) and Southern blotting of genomic DNA.

In addition to more conventional gel-electrophoresis and DNA sequencing, mutations can also be detected by in situ analysis.

Mutations in regulatory elements can also affect the level of polynucleotide (e.g., mRNA) or protein made, and can give rise to disease symptoms. Such mutations include, e.g., mutations in promoter or enhancer elements, splice signals, termination and/or polyadenylation signals; mutations which result in truncated proteins, such as chain terminators; sites involved in feed-back regulation of nucleic acid or polypeptide production; etc. Diagnostic methods to detect such mutations in regulatory elements are conventional.

In accordance with the present invention, a polynucleotide can be present in a kit, where the kit includes, e.g., one or more polynucleotides, a desired buffer (e.g., phosphate, tris, etc.), detection compositions, RNA or cDNA from different tissues to be used as controls, libraries, etc. The polynucleotide can be labeled or unlabeled, with radioactive or non-radioactive labels as known in the art. Kits can comprise one or more pairs of polynucleotides for amplifying nucleic acids specific for an alpha-7 receptor, e.g., comprising a forward and reverse primer effective in PCR. These include both sense and anti-sense orientations. For instance, in PCR-based methods (such as RT-PCR), a pair of primers are typically used, one having a sense sequence and the other having an antisense sequence.

Other Uses of Polynucleotides

The sequences of the present invention are also valuable for chromosome identification. The polynucleotides coding for the alpha-7 receptor of the invention, and homologs thereof, are specifically targeted to and can hybridize with a particular location on an individual chromosome. Moreover, there is a current need for identifying particular sites on the chromosome, for example, as part of a genome project. Thus, sequences can be mapped to chromosomes, e.g., by preparing PCR primers (preferably 15-25 bp) from the cDNA.

Fluorescence in situ hybridization (FISH) of a cDNA clone to a metaphase chromosomal spread can likewise be used to provide a precise chromosomal location in one step. This technique can be used with cDNA having at least 50 or 60 bases. For a review of this technique, see Verma et al., Human Chromosomes: a Manual of Basic Techniques, Pergamon Press, New York (1988). The chromosomal location of the alpha-7 receptor genes is known to those skilled in the art.

Once a sequence has been mapped to a precise chromosomal location, the physical position of the sequence on the chromosome can be correlated with genetic map data. Such data are found, for example, in V. McKusick, Mendelian Inheritance in Man (available on line through Johns Hopkins University Welch Medical Library). The relationship between genes and diseases that have been mapped to the same chromosomal region are then identified through linkage analysis (coinheritance of physically adjacent genes).

One can determine the differences in the cDNA or genomic sequence between affected and unaffected individuals. If a mutation is observed in some or all of the affected individuals but not in any normal individuals, then the mutation is likely to be a causative agent of the disease. With current resolution of physical mapping and genetic mapping techniques, a cDNA precisely localized to a chromosomal region associated with the disease could be one of between 50 and 500 potential causative genes. (This assumes 1 megabase mapping resolution and one gene per 20 kb).

A fragment of a polynucleotide of the present invention may also be used as a hybridization probe, e.g, for a cDNA or genomic library to isolate a full length cDNA (or genomic DNA) and to isolate other cDNAs (or genomic DNAs) which have a high sequence similarity to the gene or similar biological activity. Probes of this type preferably have at least 7 or 8 bases, more preferably about 10, 11, 12, 13, 14 15, 20, 25, etc bases, and most preferably at least about 30 bases, and exhibit about 65-100% sequence identity to part or all of the sequence coding for the novel alpha-7 receptor disclosed in FIG. 1, 3, or 5 (SEQ ID NOS: 1, 3, or 5). Such probes may also have 45 or more bases but again contain sequences which exhibit about 65-100% sequence identity to a sequence coding for some or all of the nicotinic alpha-7 receptor polypeptide of the invention, or a variant thereof. Because of the degeneracy of the genetic code, many sequences exist which exhibit a high degree of sequence identity to sequences coding for part or all of a novel alpha-7 receptor disclosed herein. The set of such sequences also includes those that code for amino acid sequences that are themselves homologous to part or all of the nicotinic alpha-7 receptor. Hybridization probes are specific to, or for, a selected polynucleotide. The phrases “specific for” or “specific to” a polynucleotide have a functional meaning that the probe can be used to identify the presence of one or more target genes in a sample. The probe is specific in the sense that it can be used to detect a polynucleotide above background noise (“non-specific binding”).

Therapeutics

The methods of the present invention are also directed to facilitating the development of potentially useful therapeutic agents that may be effective in combating alpha-7 receptor mediated or related diseases or conditions, and to methods of effecting such treatments. The invention also provides methods to enhance or restore memory function in “normal” subjects, e.g., by activating brain, especially hippocampal, neuronal alpha-7 receptors.

Any agent which modulates the expression and/or activity of an alpha-7 receptor polypeptide or polynucleotide of the invention can be used therapeutically. Some such agents are discussed elsewhere herein.

Agents which affect expression and/or activities of polypeptides of the invention can be administered to patients in need thereof by conventional procedures, in order to prevent or treat disease conditions as disclosed elsewhere herein and/or to ameliorate symptoms of those conditions. Such agents can be formulated into pharmaceutical compositions comprising pharmaceutically acceptable excipients, carriers, etc., using conventional methodologies. Formulations and excipients which enhance transfer (promote penetration) of an agent across the blood-brain barrier are also well-known in the art.

In addition to agents which can moderate the expression or activity of an alpha-7 receptor, treatment methods according to the invention also encompass the administration of an alpha-7 receptor or variant or fragment thereof to a patient in need of such therapy. For example, such a polypeptide or fragment can compensate for reduced or aberrant expression or activity of the protein, and/or, by virtue of, e.g., higher affinity for a target, can provide effective competition for it. In another embodiment, conventional methods of immunotherapy can be used.

Polynucleotides of the invention can also be used in methods of gene therapy, e.g., utilized in gene delivery vehicles. The gene delivery vehicle may be of viral or non-viral origin (see generally, Jolly, Cancer Gene Therapy 1:51-64 (1994) Kimura, Human Gene Therapy 5:845-852 (1994); Connelly, Human Gene Therapy 1:185-193 (1995); and Kaplitt, Nature Genetics 6:148-153 (1994). Gene therapy vehicles for delivery of constructs including a coding sequence of a therapeutic of the invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

The present invention can employ recombinant retroviruses which are constructed to carry or express a selected nucleic acid molecule of interest. Retrovirus vectors that can be employed include those described in EP 0 415 731; WO 90/07936; WO 94/03622; WO 93/25698; WO 93/25234; U.S. Pat. No. 5,219,740; WO 93/11230; WO 93/10218; Vile and Hart, Cancer Res. 53:3860-3864 (1993); Vile and Hart, Cancer Res. 53:962-967 (1993); Ram et al., Cancer Res. 53:83-88 (1993); Takamiya et al., J. Neurosci. Res. 33:493-503 (1992); Baba et al., J. Neurosurg. 79:729-735 (1993); U.S. Pat. No. 4,777,127; GB Patent No. 2,200,651; and EP 0 345 242. Preferred recombinant retroviruses include those described in WO 91/02805.

Packaging cell lines suitable for use with the above-described retroviral vector constructs may be readily prepared (see PCT publications WO 95/30763 and WO 92/05266), and used to create producer cell lines (also termed vector cell lines) for the production of recombinant vector particles. Within particularly preferred embodiments of the invention, packaging cell lines are made from human (such as HT1080 cells) or mink parent cell lines, thereby allowing production of recombinant retroviruses that can survive inactivation in human serum.

The present invention also employs aphavirus-based vectors that can function as gene delivery vehicles. Such vectors can be constructed from a wide variety of alphaviruses, including, for example, Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250 ATCC VR-1249; ATCC VR-532). Representative examples of such vector systems include those described in U.S. Pat. Nos. 5,091,309; 5,217,879; and 5,185,440; and PCT Publication Nos. WO 92/10578; WO 94/21792; WO 95/27069; WO 95/27044; and WO 95/07994.

Gene delivery vehicles of the present invention can also employ parvovirus such as adeno-associated virus (AAV) vectors. Representative examples include the AAV vectors disclosed by Srivastava in WO 93/09239, Samulski et al., J. Vir. 63:3822-3828 (1989); endelson et al., Virol. 166:154-165 (1988); and Flotte et al., P.N.A.S. 90:10613-10617 (1993).

Representative examples of adenoviral vectors include those described by Berkner, Biotechniques 6:616-627 (Biotechniques); Rosenfeld et al., Science 252:431-434 (1991); WO 93/19191; Kolls et al., P.N.A.S. 215-219 (1994); Kass-Eisler et al., P.N.A.S. 90:11498-11502 (1993); Guzman et al., Circulation 88:2838-2848 (1993); Guzman et al., Cir. Res. 73:1202-1207 (1993); Zabner et al., Cell 75:207-216 (1993); Li et al., Hum. Gene Ther. 4:403-409 (1993); Cailaud et al., Eur. J. Neurosci. 5: 1287-1291 (1993); Vincent et al., Nat. Genet. 5:130-134 (1993); Jaffe et al., Nat. Genet. 1:372-378 (1992); and Levrero et al., Gene 101:195-202 (1992). Exemplary adenoviral gene therapy vectors employable in this invention also include those described in WO 94/12649, WO 93/03769; WO 93/19191; WO 94/28938; WO 95/11984 and WO 95/00655. Administration of DNA linked to killed adenovirus as described in Curiel, Hum. Gene Ther. 3:147-154 (1992), may be employed.

Other gene delivery vehicles and methods may be employed, including polycationic condensed DNA linked or unlinked to killed adenovirus alone, for example, Curiel, Hum. Gene Ther. 3:147-154 (1992); ligand-linked DNA, for example, see Wu, J. Biol. Chem. 264:16985-16987 (1989); eukaryotic cell delivery vehicles cells, for example see U.S. Ser. No. 08/240,030, filed May 9, 1994, and U.S. Ser. No. 08/404,796; deposition of photopolymerized hydrogel materials; hand-held gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip, Mol. Cell Biol. 14:2411-2418 (1994) and in Woffendin, Proc. Natl. Acad. Sci. 91:1581-1585 (1994).

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by beads. The method may be improved further by treatment of the beads to increase hydrophobicity and thereby facilitate disruption of the endosome and release of the DNA into thr cytoplasm. Liposomes that can act as gene delivery vehicles are described in U.S. Pat. No. 5,422,120, PCT Patent Publication Nos. WO 95/13796, WO 94/23697 and WO 91/14445, and EP No. 0 524 968.

Further non-viral delivery suitable for use includes mechanical delivery systems such as the approach described in Woffendin et al., Proc. Natl. Acad. Sci. USA 91(24):11581-11585 (1994). Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and PCT Patent Publication No. WO 92/11033.

Computer-based Applications

The nucleotide or amino acid sequences of the invention are also provided in a variety of media to facilitate use thereof. As used herein, “provided” refers to a manufacture, other than an isolated nucleic acid or amino acid molecule, which contains a nucleotide or amino acid sequence of the present invention. Such a manufacture provides the nucleotide or amino acid sequences, or a subset thereof (e.g., a subset of open reading frames (ORFs)) in a form which allows a skilled artisan to examine the manufacture using means not directly applicable to examining the nucleotide or amino acid sequences, or a subset thereof, as they exist in nature or in purified form.

In one application of this embodiment, a nucleotide or amino acid sequence of the present invention can be recorded on computer readable media. As used herein, “computer readable media” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. The skilled artisan will readily appreciate how any of the presently known computer readable mediums can be used to create a manufacture comprising computer readable medium having recorded thereon a nucleotide or amino acid sequence of the present invention.

As used herein, “recorded” refers to a process for storing information on computer readable medium. The skilled artisan can readily adopt any of the presently known methods for recording information on computer readable medium to generate manufactures comprising the nucleotide or amino acid sequence information of the present invention.

A variety of data storage structures are available to a skilled artisan for creating a computer readable medium having recorded thereon a nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. The skilled artisan can readily adapt any number of dataprocessor structuring formats (e.g., text file or database) in order to obtain computer readable medium having recorded thereon the nucleotide sequence information of the present invention.

By providing the nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or target structural motif with the sequence information stored within the data storage means. Search means are used to identify fragments or regions of the sequences of the invention which match a particular target sequence or target motif.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. The most preferred sequence length of a target sequence is from about 10 to 100 amino acids or from about 30 to 300 nucleotide residues. However, it is well recognized that commercially important fragments, such as sequence fragments involved in gene expression and protein processing, may be of shorter length.

As used herein, “a target structural motif,” or “target motif,” refers to any rationally selected sequence or combination of sequences in which the sequence(s) are chosen on a three-dimensional configuration which is formed upon the folding of the target motif. There are a variety of target motifs known in the art. Protein target motifs include, but are not limited to, enzyme active sites and signal sequences. Nucleic acid target motifs include, but are not limited to, promoter sequences, hairpin structures and inducible expression elements (protein binding sequences).

Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the present invention. Examples of such software includes, but is not limited to, MacPattern (EMBL), BLASTN and BLASTX (NCBIA).

For example, software which implements the BLAST (Altschul et al. (1990) J. Mol. Biol. 215:403-410) and BLAZE (Brutlag et al. (1993) Comp. Chem. 17:203-207) search algorithms on a Sybase system can be used to identify open reading frames (ORFs) of the sequences of the invention which contain homology to ORFs or proteins from other libraries. Such ORFs are protein encoding fragments and are useful in producing commercially important proteins such as enzymes used in various reactions and in the production of commercially useful metabolites.

In the foregoing and in the following examples, all temperatures are set forth uncorrected in degrees Celsius; and, unless otherwise indicated, all parts and percentages are by weight.

### EXAMPLES

### Example 1

### Determination of the 5′-, 3′-ends of Rhesus Monkey Nicotinic Alpha-7 Receptor Subunit

A Gene Racer cDNA library was generated using rhesus monkey brain mRNA (Biochain) according to the standard protocol (Invitrogen). Four primers, mkα7-5′R, mkα7-5′N, mkα7-3′R and mkα7-3′N, were designed based on Genbank sequence AJ245976 and used to PCR the 5′ and 3′ ends sequences of rhesus monkey alpha 7.

RACE primers:

(SEQ ID NO: 7)

mkα7-5′R: 5′-CTCATCTCCACGCTGGCCAGGTGCAG

(SEQ ID NO: 8)

mkα7-3′R: 5′-CATGAAGAGGCCGGGAGAGGATAAGGTGCG
Nest primers:

(SEQ ID NO: 9)

mkα7-5′N: 5′-CGCACCTTATCCTCTCCCGGCCTCTTCATG

(SEQ ID NO: 10)

mkα7-3′N: 5′-CTGCACCTGGCCAGCGTGGAGATGAG

5′ and 3′ RACE PCR reactions were performed on rhesus monkey brain cDNA library using mkα7-5′R, mkα7-3′R and Gene Racer 5′-, 3′-RACE primers respectively. The PCR reactions were carried out using PCRx system and platinum HF polymerase (Invitrogen) with the following cycling characteristics: 94° C. for 3 minutes for 1 cycle; 94° C. for 30 seconds and 68° C. for 1 minute and 30 seconds for 35 cycles; 68 ° C for 7 minutes for 1 cycle. After PCR, the resulting fragments were used as templates for nested PCR using mkα7-5′N, mkα7-3′N and Gene Racer 5′-, 3′-Nest primers respectively. The nested PCR protocol was the same as the RACE PCRs.

The nested PCR products were then column purified (Qiagen) and cloned into pcDNA3.1 v5/his TOPO vector and sequenced. One clone, named mk-3′N#2, contains the 3′ end of rhesus monkey alpha 7 and is shown below. Another clone, named mkα7-5′N#13, contains the incomplete 5′ end of rhesus monkey alpha 7 and is shown below.

mk-3′N#2: the 3′ stop codon and poly A+ signal
are bolded.

(SEQ ID NO: 11)

CTGCAGCCTGGCCAGCGTGGAGATGAGCGCCGTGGCGCCGCCGCCTGCCA
GCAACGGGAACCTGCTGTACATCGGCTTCCGCGGCCTGGACGGCATGCAT
TGCGCCCCGACCCCCGACTCCGGGGTGGTGTGCGGCCGCATGGCCTGCTC
CCCCACGCACGACGAGCACCTCCTGCACGGTGGGCAGCCCCCCGAGGGGG
ACCCGGACCTGGCCAAGATCCTGGAGGAGGTCCGCTACATCGCCAACCGC
TTTCGCTGCCAGGACGAAAGCGAGGCGGTCTGCAGCGAGTGGAAGTTCGC
CGCCTGCGTGGTGGACCGCCTGTGCCTCATGGCCTTCTCGGTCTTCACCA
TCATCTGCACCATCGGCATCCTGATGTCGGCTCCCAACTTCGTGGAGGCC
GTGTCCAAAGACTTTGCGTAACCACGCCTGGTTCTGTACATGTGGAAAAC
TCACAGATGGGCAAGGCCTCTGGCTTGGTGAGATTTTGGGGTGCTAATCC
AGGACAACATTAAACGCCACAACTCCGATGTTCCCTTCTGGCTGTCAGTC
GTGTCGCTCACGGTTTCCTCATTACTTTAGGTAGTAGGATCTCAGCACTC
AGTTTAATACGCTCAGGTGGGCTGATGATCCCCTTGGCACATCCGCACTG
TCGGTCAGCAGGGCCACTGAGAAGTCATTTTGCCCATTAGCCCACTGCCT
GGAAAGCCCTTCAGAGAGCTCCCAGTGGCTCCTCACCCCGGGACAGTTGG
TTTTGCATGTCTGCATGCCACTTGCCATGAAGGCCTACCTGAAAATTCAA
CATTTGCTTTTTGCTTGTGTACAAACCTAGATTGAAGCTAAAATAAACCA
GACTCACTAAATCCAAAAAAAAAAAAAAAA
mkα7-5′N#13:

(SEQ ID NO: 12)

GTATTTTGAGCGCGTCTCGATCAGCTTTCGTTTCAGTCTTCTGTTTCCGT
CACCCACACGGGCATATTCAAGAGTTCCTGCTACATCGACGTGCGCCGGT
TTCCCTTTGATGTGCAGCATTGCAAACTGAAGTTTGGATCCTGGTCTTAT
GGAGGCTGGTCCTTGGATCTGCAGATGCAGGAGGCAGATATCAGTGGCTA
TATCCCCAGTGGAGAATGGGACCTAGTGGGAATTCCCGGCAAGAGGAGTG
AAAAGTTCTATGAGTGCTGCAAAGAGCCCTACCCCGATGTCACCTTCACA
GTGACCATGCGCCGCAGGACCCTCTACTACGGCCTCAACCTGCTGATCCC
CTGTGTGCTCATCTCTGCCCTTGCCCTGCTGGTGTTCCTGCTTCCTGCAG
ATTCCGGGGAGAAGATTTCCCTGGGGATAACAGTCTTACTCTCTCTCACT
GTCTTCATGCTGCTCGTGGCTGAGATCATGCCCGCAACATCTGATTCAGT
ACCATTGATAGCCCAGTACTTCGCCAGCACCATGATCATCGTGGGCCTCT
CCGTGGTGGTGACGGTGATCGTGCTGCAGTACCACCACCACGACCCCGAC
GGGGGCAAGATGCCCAAGTGGACCAGAGTCATCCTTCTGAACTGGTGCGC
GTGGTTCCTGCGCATGAAGAGGCCGGGAGAGGATAAGGTGCG

To obtain the complete 5′ end sequence of rhesus monkey alpha 7, two primers, mkα7-5′R1 and mkα7-5′N1, were designed according to the sequence of mkα7-5′N#13 and used to PCR the same cDNA library as described above, yet with the following cycling characteristics: 94° C. for 3 minutes for 1 cycle; 94° C. for 30 seconds, 65° C. for 30 seconds and 68° C. for 1 minute for 35 cycles; 68° C. for 7 minutes for 1 cycle. The nested PCR products were then column purified (Qiagen) and cloned into pcDNA3.1 v5/his TOPO vector and sequenced.

RACE primer

(SEQ ID NO: 13)

mkα7-5′R1: 5′-GACCAGCCTCCATAAGACCAGGATCCAAACTTCAG
Nest primer

(SEQ ID NO: 14)

mkα7-5′N1: 5′-CGCACGTCGATGTAGCAGGAACTCTTGAATATGC

One clone, named mkα7#1, still only contained the incomplete 5′ end of rhesus monkey alpha-7 and is shown below.

mkα7#1:

(SEQ ID NO: 15)

CATTGGCGGCATCTGTCCTCCCCGACAGGGTGCCTCCAGCACTTCAGATC
CCAGCCGAGAGTCTGGCTGCTAGCGCCCAGCAAACGTGTCCCTGCAAGGC
GAGTTCCAGAGGAAGCTTTACAAGGAGCTGGTCAAGAACTACAACCCCTT
GGAGAGGCCCGTGGCCAATGACTCGCAACCGCTCACCGTCTACTTCTCCC
TGAGCCTCCTGCAGATCATGGACGCGGATGAGAAGAACCAAGTTTTAACC
ACCAACATTTGGCTGCAAATGTCTTGGACAGATCACTATTTACAGTGGAT
GTGTCAGAATATCCAGGGGTGAAGACTGTTCGTTTCCCAGATGGCCAGAT
TTGGAAACCAGACATTCTTCTCTATAACAGTGCGGATGAGCGCTTTGACG
CCACATTCCACACCAACGTGTTGGTGAATTCTTCTGGGCATTGCCAGTAC
CTGCCTCCAGGCATATTCAAGAGTTCCTGCTAATCGACGTGCG

Again, to obtain the complete 5′ end sequence of rhesus monkey alpha-7, two more primers, mkα7-5′R2 and mkα7-5′N2, were designed according to the sequence of mkα7#1 and used to PCR the same cDNA library as described above, yet with the following cycling characteristics: 94° C. for 5 minutes for 1 cycle; 94° C. for 20 seconds, 65° C. for 20 seconds and 68° C. for 30 seconds for 35 cycles; 68° C. for 7 minutes for 1 cycle. The nested PCR products were then column purified (Qiagen) and cloned into pcDNA3.1 v5/his TOPO vector and sequenced.

One clone, named mkα7-5′N#16, contains the 5′ Met of rhesus monkey alpha 7 and is shown below.

mkα7-5′N#16: the starting Met is bolded

(SEQ ID NO: 16)

GAGAGGCGGCTCTGTGGCCACAGGCGCAGGCCCGGGCGACAGCCGATACG
TGAGGCGCGCCGGCCCGCGGCAGCTCCGGGACTCAACATGCGCTGCTCGC
AGGGAGGCGTCTGGCTGGCTCTGGCCGCGTCGCTCCTGCATGTGTCCCTG
CAAGGCGAGTTCCAGAGGAAGCTTTACAAGGAGCTGGTCAAGAACTACAA
CCCCTTGGAGAGGCCCGTGGCCAATGACTCGCAACCGCTCACCGTCTAC

### Full-length Cloning of Rhesus Monkey Nicotinic Alpha-7 Subunit

Two primers, mkα7-5′b and mkα7-3′a, were designed based on the sequences of mKα7-5′N#16 and mk-3′N#2 (see above) and used to PCR full-length rhesus monkey alpha-7 from the same cDNA library.

(SEQ ID NO: 17)

mkα7-5′b: 5′-CTCAACATGCGCTGCTCGCAGGGAGG

(SEQ ID NO: 18)

mkα7-3′a: 5′-CCAAGCCAGAGGCCTTGCCCATCTGTGAG

The PCR reaction was performed as described above with the following cycling characteristics: 94° C. for 5′ for 1 cycle; 94° C. for 30 seconds, 65° C. for 30 seconds and 68° C. for 2 minutes for 35 cycles; 68° C. for 7 minutes for 1 cycle. The resulting PCR fragment (˜1.6 kb) was column purified (Qiagen) and cloned into pcDNA3.1 v5/his TOPO vector and sequenced.

One clone, named mkalpha7#11, contained the full-length cDNA sequence of rhesus monkey alpha 7. The cDNA and protein sequences are shown in FIG. 1 (SEQ ID NO: 1) and FIG. 2 (SEQ ID NO: 2), respectively. This clone, with the 5′ Met of the full-length rhesus monkey nicotinic alpha-7 is downstream of the vector CMV promoter, can be used to establish a stable cell line to expressed the recombinant receptor.

### Example 2

### Generating a Mutant Rhesus Monkey Alpha 7, mkalpha7(L270T)

To mutagenize rhesus monkey alpha 7 at amino acid position 270, the primers shown below were used to PCR mkalpha7#11, according to the protocol of Quick-Change kit (Stratagene). After the reaction, the PCR products were treated with DpnI at 37° C. for one hour. After treatment, the remaining products were used to transform Top10 competent cells (Invitrogen). Plasmid DNAs from three individual clones were sequenced to detect mutation L270T. The nucleic acid and protein sequences of one mutant clone, named mka7L270T#3, are shown in FIG. 3 (SEQ ID NO: 3) and FIG. 4 (SEQ ID NO: 4), respectively.

Primers:

(SEQ ID NO: 19)

mkα7-14: 5′-GGGATAACAGTCACTTCTCTCACTGTCTTC

(SEQ ID NO: 20)

mkα7-15: 5′-GAAGACAGTGAGAGAAGTGACTGTTATCCC

### Generating a Double-mutant Rhesus Monkey Alpha 7, mkalpha7(L270T/S193N)

To mutagenize rhesus monkey alpha 7 at amino acid position 193, in addition to position 270, the following primers (see below) were used to PCR mka7L270T#3, according to the protocol of Quick-Change kit (Stratagene). After the reaction, the PCR products were treated with DpnI at 37° C. for one hour. After treatment, the remaining products were used to transform Top10 competent cells (Invitrogen). Plasmid DNAs from three individual clones were sequenced to detect mutation S193N. The sequences of one double-mutant clone, named mka7L270T/S193N#1, are shown in FIGS. 5 and 6.

mkalpha7S193N-5′:

(SEQ ID NO: 21)

5′-AGTGGCTATATCCCCAACGGAGAATGGGACCTAG 3′
mkalpha7S193N-3′:

(SEQ ID NO: 22)

5′-CTAGGTCCCATTCTCCGTTGGGGATATAGCCAC 3′

### Example 3

### Cell Culture and Transfections

QM 7 cells were routinely grown as monolayers in minimum essential medium 199 supplemented with 5% FBS, 2% tryptose phosphate broth, 1% DMSO, penicillin (100 IU/ml) and were passaged every 3-4 days. Transfections with either rhesus monkey wild type alpha7 nAchR (mkα7) or mutant alpha7 nAchR (mkα7/L270T) were performed using Lipofectamine PLUS Kit (Invitrogen). Twenty-four hours after the transfection, cells were split for stable selection with 1 mg/ml G418. Single colonies were isolated and propagated. The expression of mkα7/L270T was identified with FLIPR for fluorescence changes after addition of agonist. The expression of mkα7 was identified by radioligand binding to [³H]-methyllycaconitine (Virginio, C., A., Giacometti, A. et al. (2002). Pharmacological properties of rat alpha7 nicotinic receptors expressed in native and recombinant cell systems, Eur, J. Pharmacol. 445(3), 153-161.)

The double-mutant rhesus monkey alpha 7, mkalpha7(L270T/S 193N), containing plasmids were transiently transfected into QM-7 cells with Lipofectamine PLUS reagent (Invitrogen), in 175 cm² flasks. Twenty four hours after the transfection, the cells were collected and re-seeded into a 96-well plate at a density of 40,000 cells/well for functional measurement.

### Example 4

### Functional Measurement of Mutant (L270T) mkα7/L270T Receptor and Double Mutant mkalpha7(L270T/S193N) Using FLIPR

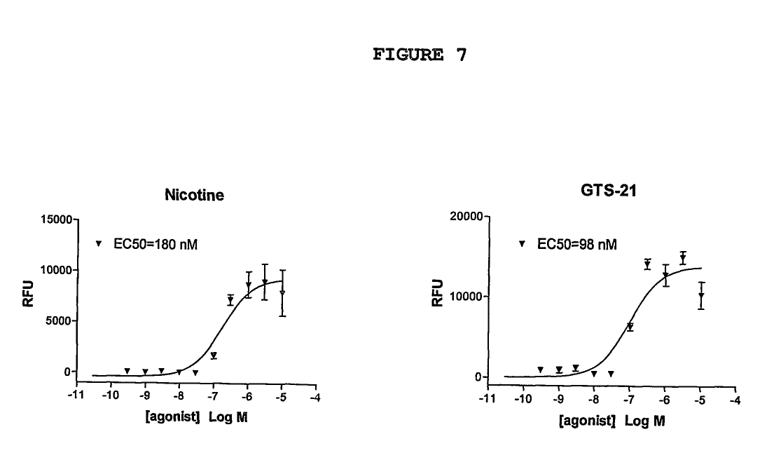

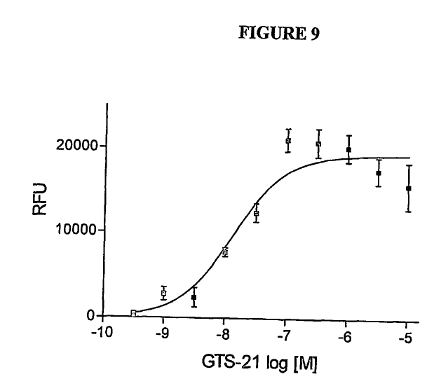

Cells were seeded into 96 well black wall and clear-bottom plates (Costar) coated with poly-D-lysine at a density of 20,000 cells per well and cultured overnight. On the second day, the cells were incubated with 1×HBSS, 20 mM HEPES, 2.5 mM Probenecid (Sigma), 0.1% BSA, pH 7.4 buffer (FLIPR buffer) in the presence of Fluo-3AM (Molecular Probe) at 37° C. for 60 min. After washing three times to remove the Fluo-3AM, FLIPR buffer was added into the cell plates. The plates were placed onto a FLIPR (Molecular Devices). Compound addition was performed by FLIPR pipetting system. The fluorescence was monitored (λₑₓ=488 nM, λ_{EM}=540 nM) for three minutes immediately after the compound addition. The relative fluorescence unit (RFU) was measured as peak fluorescence intensity minus basal fluorescence intensity. Curve fitting and parameter estimation were carried out using Graph Pad Prism 3.00 (GraphPad Software Inc., California, U.S.A.) The responses to nicotine and GTS-21, nicotinic alpha 7 agonists, for mkα7/L270T Receptor were tested and shown in FIG. 7. The responses of double mutant mka (L270T/S 193N) to GTS-21, a nicotinic alpha 7 agonist, were tested and shown in FIG. 9.

### Example 5

### Functional Measurement of mkα7 Using Patch Clamp Method

Recording

Stably transfected QM7 cells grown on cover slips and expressing wild-type rhesus monkey alpha7 receptors were incubated at 37° C. in the presence of 5% CO₂. Individual cover slips were removed for function analysis and placed in a recording buffer consisting of 140 mM NaCl, 5 mM KCl, 10 mM HEPES, 2 mM CaCl₂, 1 mM MgCl₂, 100 mM glucose titrated to pH 7.3 and perfused at 2-3 ml/min. Borosilicate recording pipettes were filled with a solution consisting of 140 mM CsCl, 10 mM HEPES, 4 mM NaCl, 4 mM MgCl₂, 2 mM CaCl₂, 5 mM EGTA titrated to pH 7.3. For whole cell recording, pipettes with resistances between 3.0-8.0 MΩ were used. Upon gigaseal formation, holding potential was set to −60 mV and the cell was ruptured by suction. Cell is held at −60 mV at all times except where noted below.

Drug Application

DAD-VC perfusion system by ALA Scientific Instruments was modified to include an internally perfused pipette to rapidly and precisely deliver drug (based on Kabokov and Papke, 1998). Briefly, drug flows within the pipette and out to a waste receptacle by gravity. By closing the valve that leads to the waste container, the drug is forced out of the pipette onto the cell. Prior to drug application, the cell is hyperpolarized to −100 mV for a duration of 500 ms. During the hyperpolarization, drug is rapidly dispensed for 1.5 seconds. Recording continues at −100 mV for another 4 seconds before returning the cell to the holding potential of 60 mV. Upon completion of recording, the perfusion system primes the pipette with the next dosage. An intersweep interval equal to 65 seconds is sufficient to completely exchange the contents of the perfusion pipette. The cell is initially exposed to a concentration of 1 mM Acetylcholine (ACh), which maximally activates alpha7 receptors (Papke and Porter Papke, 2002). Two experimental drug concentrations follow and the protocol concludes with one final dose of 1 mM ACh.

Data Analysis

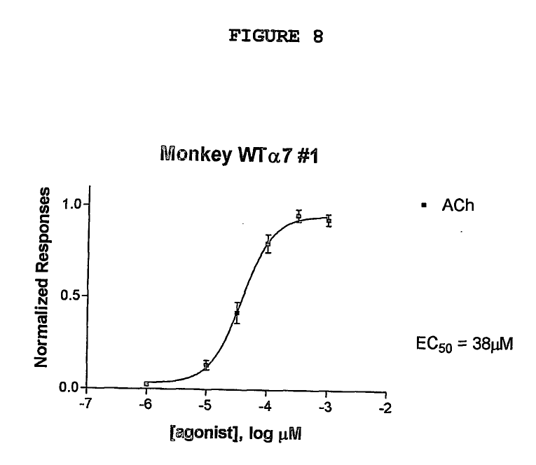

Net charge analysis is used to assess the total amount of charge flowing through the channel in response to receptor agonism. Net charge analysis is believed to give a better indication of the nicotinic alpha-7 receptor than more traditional peak measurements (Papke and Porter Papke, 2002). Each experimental concentration of drug is compared to the cell's initial response to 1 mM ACh. The final Ach response is also used to account for any receptor desensitization. All experimental values are therefore normalized to the cell's maximum response to 1 mM ACh. These normalized values are used to generate a dose response curve that reflects the efficacy of the alpha7 receptor to either a known or experimental drug (FIG. 8).

The topic headings set forth above are meant as guidance as to where certain information can be found in the application. They are not intended to be the only source in the application where information on such a topic can be found.

From the foregoing description, one skilled in the art can easily ascertain the essential characteristics of this invention, and without departing from the spirit and scope thereof, can make changes and modifications of the invention to adapt it to various usage and conditions.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosure of all applications, patents and publications, cited above and in the figures are hereby incorporated in their entirety by reference, including U.S. Provisional Application Ser. Nos. 60/447,288, filed Feb. 14, 2003, and 60/453,204, filed Mar. 11, 2003

1. An isolated polynucleotide for a monkey alpha-7 nicotinic receptor, or a mutation thereof.

2. An isolated polynucleotide for an alpha-7 nicotinic acetylcholine receptor, comprising:
(a) a polynucleotide sequence set forth in FIG. 1 (SEQ ID NO: 1),
(b) a polynucleotide sequence coding for a polypeptide having the amino acid sequence from amino acid 1-502 or 23-502 as set forth in FIG. 2 (SEQ ID NO: 2),
(c) a polynucleotide sequence coding for a polypeptide having one or more amino acid substitutions in the M2 region of the polypeptide sequence set forth in FIG. 2 (SEQ ID NO: 2) from amino acid 1-502 or 23-502, or
complements thereto.

3. An isolated polynucleotide of claim 1, which codes without interruption for said alpha-7 nicotinic acetylcholine receptor.

4. An isolated polynucleotide of claim 1, comprising a polynucleotide sequence coding for a polypeptide having one or more of the following amino acid substitutions: L270T, L270S, L270F, L270V, V274T, T267Q, E260A/V274T, E260A/L270T, E260A/L270S, E260A/L270V, E260A/L270F, E260A/T267Q, E260A/L277T, and/or E260A/L278T.

5. An isolated polynucleotide of claim 4, comprising a polynucleotide sequence set forth in FIG. 3 (SEQ ID NO : 3), or a polynucleotide sequence coding for a polypeptide comprising an amino acid substitution L270T as set forth in FIG. 4 (SEQ ID NO : 4).

6. An isolated polynucleotide of claim 1 or 2, comprising a polynucleotide sequence coding for a polypeptide comprising one or more of the following amino acid substitutions: A65V, R156W, S193N, K208R, K208S, M395V, A398V or A398T.

7. An isolated polynucleotide of claim 6, comprising a polynucleotide sequence set forth in FIG. 5 (SEQ ID NO: 5), or a polynucleotide sequence coding for a polypeptide comprising amino acid substitutions L270T and S193N as set forth in FIG. 6 (SEQ ID NO: 6).

8. An isolated polynucleotide of claim 1, wherein said polynucleotide has the complete polynucleotide sequence coding for monkey alpha-7 nicotinic receptor of the cDNA clone contained in the plasmid deposited with the ATCC as deposit PTA-5004.

9. An expression vector, comprising: a polynucleotide of claim 1 or 2 operably linked to a promoter sequence.

10. A transfected host cell, comprising: a polynucleotide of claim 1 or 2.

11. A transfected host cell, comprising: an expression vector comprising a polynucleotide of claim 1 or 2 operably linked to a promoter sequence.

12. A transfected host cell of claim 9, wherein the alpha-7 nicotinic acetylcholine receptor coded for by said polynucleotide is expressed on the surface membrane of said host cell.

13. A transfected host cell of claim 8, wherein said cell is a QM7 or QT6 cell.

14. An isolated polypeptide which is coded for a polynucleotide sequence of claim 1 or 2.

15. A method for identifying an agent that modulates the expression or activity of an alpha-7 nicotinic acetylcholine receptor in transfected host cells, comprising:
contacting a transfected host cell of claim 10 with a test agent under conditions effective for said test agent to modulate the expression or activity of said alpha-7 nicotinic acetylcholine receptor, wherein said receptor is expressed on the surface membrane of said cell, and
determining whether said test agent modulates the expression or activity of said alpha- 7 nicotinic acetylcholine receptor.

16. A method of claim 15, wherein said determining comprises: measuring the total amount of charge flowing across the membrane of said cell, or measuring the change in a calcium-sensitive dye present in said cell, in response to said agent.

17. A method of claim 15, wherein the agent activates the expression or activity of said receptor.

18. An antibody which is specific for a polypeptide which is coded for by a polynucleotide of claim 1 or 2.

19. A non-human transgenic mammal comprising, a polynucleotide of claim 1 or 2 coding for a monkey alpha-7 nicotinic receptor.

20. A mammalian cell whose genome comprises a functional disruption of the endogenous monkey alpha-7 nicotinic receptor encoding a polynucleotide of claim 1 or 2.

21. A method of selecting a polynucleotide sequence or polypeptide coding for an alpha-7 nicotinic acetylcholine receptor from a database comprising polynucleotide or polypeptide sequences, comprising
displaying, in a computer-readable medium, a polynucleotide sequence or polypeptide sequence of claim 1 or 2, wherein said displayed sequences have been retrieved from said database upon selection by a user.


SEQUENCE LISTING (22 sequences)

SEQ ID NO: 1 - DNA, 1571 bp, Macaca mulatta
  CDS (7)..(1515)
ctcaacatgcgctgctcgcagggaggcgtctggctggctctggccgcgtcgctcctgcatgtgtccctgcaaggcgagttccagaggaagctttacaaggagctggtcaagaactacaaccccttggagaggcccgtggccaatgactcgcaaccgctcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgcggatgagcgctttgacgccacattccacaccaacgtgttggtgaattcttctgggcattgccagtacctgcctccaggcatattcaagagttcctgctacatcgacgtgcgctggtttccctttgatgtgcagcattgcaaactgaagtttggatcctggtcttatggaggctggtccttggatctgcagatgcaggaggcagatatcagtggctatatccccagtggagaatgggacctagtgggaattcccggcaagaggagtgaaaagttctatgagtgctgcaaagagccctaccccgatgtcaccttcacagtgaccatgcgccgcaggaccctctactacggcctcaacctgctgatcccctgtgtgctcatctctgcccttgccctgctggtgttcctgcttcctgcagattccggggagaagatttccctggggataacagtcttactctctctcactgtcttcatgctgctcgtggctgagatcatgcccgcaacatctgattcagtaccattgatagcccagtacttcgccagcaccatgatcatcgtgggcctctcggtggtggtgacggtgatcgtgctgcagtaccaccaccacgaccccgacgggggcaagatgcccaagtggaccagagtcatccttctgaactggtgcgcgtggttcctgcgcatgaagaggcccggagaggataaggtgcgcccggcctgccagcacaagcagcgccgctgcagcctggccagcgtggagatgagcgccgtggcgccgccgcctgccagcaacgggaacctgctgtacatcggcttccgcggcctggacggcatgcattgcgccccgacccccgactccggggtggtgtgcggccgcatggcctgctcccccacgcacgacgagcacctcctgcacggtgggcagccccccgagggggacccggacctggccaagatcctggaggaggtccgctacatcgccaaccgctttcgctgccaggacgaaagcgaggcggtctgcagcgagtggaagttcgccgcctgcgtggtggaccgcctgtgcctcatggccttctcggtcttcaccatcatctgcaccatcggcatcctgatgtcggctcccaacttcgtggaggccgtgtccaaagactttgcgtaaccacgcctggttctgtacatgtggaaaactcacagatgggcaaggcctctggcttg

SEQ ID NO: 2 - protein, 502 aa, Macaca mulatta
MRCSQGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPSGEWDLVGIPGKRSEKFYECCKEPYPDVTFTVTMRRRTLYYGLNLLIPCVLISALALLVFLLPADSGEKISLGITVLLSLTVFMLLVAEIMPATSDSVPLIAQYFASTMIIVGLSVVVTVIVLQYHHHDPDGGKMPKWTRVILLNWCAWFLRMKRPGEDKVRPACQHKQRRCSLASVEMSAVAPPPASNGNLLYIGFRGLDGMHCAPTPDSGVVCGRMACSPTHDEHLLHGGQPPEGDPDLAKILEEVRYIANRFRCQDESEAVCSEWKFAACVVDRLCLMAFSVFTIICTIGILMSAPNFVEAVSKDFA

SEQ ID NO: 3 - DNA, 1571 bp, Macaca mulatta
  CDS (7)..(1515)
ctcaacatgcgctgctcgcagggaggcgtctggctggctctggccgcgtcgctcctgcatgtgtccctgcaaggcgagttccagaggaagctttacaaggagctggtcaagaactacaaccccttggagaggcccgtggccaatgactcgcaaccgctcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgcggatgagcgctttgacgccacattccacaccaacgtgttggtgaattcttctgggcattgccagtacctgcctccaggcatattcaagagttcctgctacatcgacgtgcgctggtttccctttgatgtgcagcattgcaaactgaagtttggatcctggtcttatggaggctggtccttggatctgcagatgcaggaggcagatatcagtggctatatccccagtggagaatgggacctagtgggaattcccggcaagaggagtgaaaagttctatgagtgctgcaaagagccctaccccgatgtcaccttcacagtgaccatgcgccgcaggaccctctactacggcctcaacctgctgatcccctgtgtgctcatctctgcccttgccctgctggtgttcctgcttcctgcagattccggggagaagatttccctggggataacagtcttaacttctctcactgtcttcatgctgctcgtggctgagatcatgcccgcaacatctgattcagtaccattgatagcccagtacttcgccagcaccatgatcatcgtgggcctctcggtggtggtgacggtgatcgtgctgcagtaccaccaccacgaccccgacgggggcaagatgcccaagtggaccagagtcatccttctgaactggtgcgcgtggttcctgcgcatgaagaggcccggagaggataaggtgcgcccggcctgccagcacaagcagcgccgctgcagcctggccagcgtggagatgagcgccgtggcgccgccgcctgccagcaacgggaacctgctgtacatcggcttccgcggcctggacggcatgcattgcgccccgacccccgactccggggtggtgtgcggccgcatggcctgctcccccacgcacgacgagcacctcctgcacggtgggcagccccccgagggggacccggacctggccaagatcctggaggaggtccgctacatcgccaaccgctttcgctgccaggacgaaagcgaggcggtctgcagcgagtggaagttcgccgcctgcgtggtggaccgcctgtgcctcatggccttctcggtcttcaccatcatctgcaccatcggcatcctgatgtcggctcccaacttcgtggaggccgtgtccaaagactttgcgtaaccacgcctggttctgtacatgtggaaaactcacagatgggcaaggcctctggcttg

SEQ ID NO: 4 - protein, 502 aa, Macaca mulatta
MRCSQGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPSGEWDLVGIPGKRSEKFYECCKEPYPDVTFTVTMRRRTLYYGLNLLIPCVLISALALLVFLLPADSGEKISLGITVLTSLTVFMLLVAEIMPATSDSVPLIAQYFASTMIIVGLSVVVTVIVLQYHHHDPDGGKMPKWTRVILLNWCAWFLRMKRPGEDKVRPACQHKQRRCSLASVEMSAVAPPPASNGNLLYIGFRGLDGMHCAPTPDSGVVCGRMACSPTHDEHLLHGGQPPEGDPDLAKILEEVRYIANRFRCQDESEAVCSEWKFAACVVDRLCLMAFSVFTIICTIGILMSAPNFVEAVSKDFA

SEQ ID NO: 5 - DNA, 1571 bp, Macaca mulatta
  CDS (7)..(1515)
ctcaacatgcgctgctcgcagggaggcgtctggctggctctggccgcgtcgctcctgcatgtgtccctgcaaggcgagttccagaggaagctttacaaggagctggtcaagaactacaaccccttggagaggcccgtggccaatgactcgcaaccgctcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgcggatgagcgctttgacgccacattccacaccaacgtgttggtgaattcttctgggcattgccagtacctgcctccaggcatattcaagagttcctgctacatcgacgtgcgctggtttccctttgatgtgcagcattgcaaactgaagtttggatcctggtcttatggaggctggtccttggatctgcagatgcaggaggcagatatcagtggctatatccccaacggagaatgggacctagtgggaattcccggcaagaggagtgaaaagttctatgagtgctgcaaagagccctaccccgatgtcaccttcacagtgaccatgcgccgcaggaccctctactacggcctcaacctgctgatcccctgtgtgctcatctctgcccttgccctgctggtgttcctgcttcctgcagattccggggagaagatttccctggggataacagtcttaacttctctcactgtcttcatgctgctcgtggctgagatcatgcccgcaacatctgattcagtaccattgatagcccagtacttcgccagcaccatgatcatcgtgggcctctcggtggtggtgacggtgatcgtgctgcagtaccaccaccacgaccccgacgggggcaagatgcccaagtggaccagagtcatccttctgaactggtgcgcgtggttcctgcgcatgaagaggcccggagaggataaggtgcgcccggcctgccagcacaagcagcgccgctgcagcctggccagcgtggagatgagcgccgtggcgccgccgcctgccagcaacgggaacctgctgtacatcggcttccgcggcctggacggcatgcattgcgccccgacccccgactccggggtggtgtgcggccgcatggcctgctcccccacgcacgacgagcacctcctgcacggtgggcagccccccgagggggacccggacctggccaagatcctggaggaggtccgctacatcgccaaccgctttcgctgccaggacgaaagcgaggcggtctgcagcgagtggaagttcgccgcctgcgtggtggaccgcctgtgcctcatggccttctcggtcttcaccatcatctgcaccatcggcatcctgatgtcggctcccaacttcgtggaggccgtgtccaaagactttgcgtaaccacgcctggttctgtacatgtggaaaactcacagatgggcaaggcctctggcttg

SEQ ID NO: 6 - protein, 502 aa, Macaca mulatta
MRCSQGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSEKFYECCKEPYPDVTFTVTMRRRTLYYGLNLLIPCVLISALALLVFLLPADSGEKISLGITVLTSLTVFMLLVAEIMPATSDSVPLIAQYFASTMIIVGLSVVVTVIVLQYHHHDPDGGKMPKWTRVILLNWCAWFLRMKRPGEDKVRPACQHKQRRCSLASVEMSAVAPPPASNGNLLYIGFRGLDGMHCAPTPDSGVVCGRMACSPTHDEHLLHGGQPPEGDPDLAKILEEVRYIANRFRCQDESEAVCSEWKFAACVVDRLCLMAFSVFTIICTIGILMSAPNFVEAVSKDFA

SEQ ID NO: 7 - DNA, 26 bp, Macaca mulatta
ctcatctccacgctggccaggtgcag

SEQ ID NO: 8 - DNA, 30 bp, Macaca mulatta
catgaagaggccgggagaggataaggtgcg

SEQ ID NO: 9 - DNA, 30 bp, Macaca mulatta
cgcaccttatcctctcccggcctcttcatg

SEQ ID NO: 10 - DNA, 26 bp, Macaca mulatta
ctgcacctggccagcgtggagatgag

SEQ ID NO: 11 - DNA, 880 bp, Macaca mulatta
ctgcagcctggccagcgtggagatgagcgccgtggcgccgccgcctgccagcaacgggaacctgctgtacatcggcttccgcggcctggacggcatgcattgcgccccgacccccgactccggggtggtgtgcggccgcatggcctgctcccccacgcacgacgagcacctcctgcacggtgggcagccccccgagggggacccggacctggccaagatcctggaggaggtccgctacatcgccaaccgctttcgctgccaggacgaaagcgaggcggtctgcagcgagtggaagttcgccgcctgcgtggtggaccgcctgtgcctcatggccttctcggtcttcaccatcatctgcaccatcggcatcctgatgtcggctcccaacttcgtggaggccgtgtccaaagactttgcgtaaccacgcctggttctgtacatgtggaaaactcacagatgggcaaggcctctggcttggtgagattttggggtgctaatccaggacaacattaaacgccacaactccgatgttcccttctggctgtcagtcgtgtcgctcacggtttcctcattactttaggtagtaggatctcagcactcagtttaatacgctcaggtgggctgatgatccccttggcacatccgcactgtcggtcagcagggccactgagaagtcattttgcccattagcccactgcctggaaagcccttcagagagctcccagtggctcctcaccccgggacagttggttttgcatgtctgcatgccacttgccatgaaggcctacctgaaaattcaacatttgctttttgcttgtgtacaaacctagattgaagctaaaataaaccagactcactaaatccaaaaaaaaaaaaaaaa

SEQ ID NO: 12 - DNA, 692 bp, Macaca mulatta
gtattttgagcgcgtctcgatcagctttcgtttcagtcttctgtttccgtcacccacacgggcatattcaagagttcctgctacatcgacgtgcgccggtttccctttgatgtgcagcattgcaaactgaagtttggatcctggtcttatggaggctggtccttggatctgcagatgcaggaggcagatatcagtggctatatccccagtggagaatgggacctagtgggaattcccggcaagaggagtgaaaagttctatgagtgctgcaaagagccctaccccgatgtcaccttcacagtgaccatgcgccgcaggaccctctactacggcctcaacctgctgatcccctgtgtgctcatctctgcccttgccctgctggtgttcctgcttcctgcagattccggggagaagatttccctggggataacagtcttactctctctcactgtcttcatgctgctcgtggctgagatcatgcccgcaacatctgattcagtaccattgatagcccagtacttcgccagcaccatgatcatcgtgggcctctccgtggtggtgacggtgatcgtgctgcagtaccaccaccacgaccccgacgggggcaagatgcccaagtggaccagagtcatccttctgaactggtgcgcgtggttcctgcgcatgaagaggccgggagaggataaggtgcg

SEQ ID NO: 13 - DNA, 35 bp, Macaca mulatta
gaccagcctccataagaccaggatccaaacttcag

SEQ ID NO: 14 - DNA, 34 bp, Macaca mulatta
cgcacgtcgatgtagcaggaactcttgaatatgc

SEQ ID NO: 15 - DNA, 494 bp, Macaca mulatta
cattggcggcatctgtcctccccgacagggtgcctccagcacttcagatcccagccgagagtctggctgctagcgcccagcaaacgtgtccctgcaaggcgagttccagaggaagctttacaaggagctggtcaagaactacaaccccttggagaggcccgtggccaatgactcgcaaccgctcaccgtctacttctccctgagcctcctgcagatcatggacgcggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgcggatgagcgctttgacgccacattccacaccaacgtgttggtgaattcttctgggcattgccagtacctgcctccaggcatattcaagagttcctgctaatcgacgtgcg

SEQ ID NO: 16 - DNA, 248 bp, Macaca mulatta
gagaggcggctctgtggccacaggcgcaggcccgggcgacagccgatacgtgaggcgcgccggcccgcggcagctccgggactcaacatgcgctgctcgcagggaggcgtctggctggctctggccgcgtcgctcctgcatgtgtccctgcaaggcgagttccagaggaagctttacaaggagctggtcaagaactacaaccccttggagaggcccgtggccaatgactcgcaaccgctcaccgtcta

SEQ ID NO: 17 - DNA, 26 bp, Macaca mulatta
ctcaacatgcgctgctcgcagggagg

SEQ ID NO: 18 - DNA, 29 bp, Macaca mulatta
ccaagccagaggccttgcccatctgtgag

SEQ ID NO: 19 - DNA, 30 bp, Macaca mulatta
gggataacagtcacttctctcactgtcttc

SEQ ID NO: 20 - DNA, 30 bp, Macaca mulatta
gaagacagtgagagaagtgactgttatccc

SEQ ID NO: 21 - DNA, 34 bp, Macaca mulatta
agtggctatatccccaacggagaatgggacctag

SEQ ID NO: 22 - DNA, 33 bp, Macaca mulatta
ctaggtcccattctccgttggggatatagccac